(19) United States
(12) Patent Application Publication
(10) Pub. No.: US 2015/0182703 A1
(43) Pub. Date: Jul. 2, 2015

(54) NANOFLUIDIC DELIVERY SYSTEM

(71) Applicant: Biltmore Technologies, Inc., Palm Beach, FL (US)

(72) Inventors: Troy G. Fohrman, Palm City, FL (US); David Carnahan, Needham, MA (US); Nolan Nicholas, Worcester, MA (US); Howard Busch, Lantana, FL (US); Tyler J. Fohrman, Palm City, FL (US); Keith Boudreau, Palm Beach, FL (US)

(21) Appl. No.: 14/558,485

(22) Filed: Dec. 2, 2014

Related U.S. Application Data
(60) Provisional application No. 61/910,486, filed on Dec. 2, 2013.
(60) Provisional application No. 61/910,491, filed on Dec. 2, 2013.

(51) Int. Cl. A61M 5/32; A61M 37/00

(52) U.S. Cl. CPC A61M 5/32; A61M 5/3295; A61M 37/0015; A61M 2037/0023; A61M 2037/003; A61M 2037/0061

(57) ABSTRACT

Painless, non-invasive nanofluidic delivery systems useful for the delivery of therapeutically active agents, GRAS agents, sterile fluids and inert fillers through epithelial membranes are described. The painless, non-invasive delivery system delivers a therapeutic agent via a micro-syringe-based assembly. The painless, non-invasive delivery system may also have a vehicle, which facilitates the absorption of the therapeutic agents by altering its absorption rate at the site of administration. Also disclosed is a method and components of the delivery system to administer therapeutic agents in a consistent, predictable and reproducible manner. Certain compositions may facilitate the delivery of different agents without altering the agents from their current or previous form.

### REFERENCE TO PENDING PRIOR PATENT APPLICATION

This patent application:

(i) claims benefit of pending prior U.S. Provisional Patent Application Ser. No. 61/910,486, filed Dec. 2, 2013 by Paradox Private Equity Funds, LLC and Troy G. Fohrman et al. for NANOFLUIDIC DELIVERY SYSTEM (Attorney's Docket No. FOHRMAN-1 PROV); and

(ii) claims benefit of pending prior U.S. Provisional Patent Application Ser. No. 61/910,491, filed Dec. 2, 2013 by Paradox Private Equity Funds, LLC and David Carnahan et al. for NANOFLUIDIC DELIVERY SYSTEM (Attorney's Docket No. FOHRMAN-2 PROV).

The two (2) above-identified patent applications are hereby incorporated herein by reference.

### FIELD OF THE INVENTION

This invention generally relates to the use of new high-strength nanostructures of carbon or similar materials to transfer (i.e., deliver) pure fluids and ingredient-doped fluids into mammals via nanosyringes using a novel biophysical process sometimes referred to as eleutherospherin nanofluidics; more particularly, this invention relates to novel processes and systems for the unconjugated transport of various small molecules and/or biological and/or pharmacologically-active ingredients via the dermal tissues; and most particularly, this invention relates to methods and apparatus for the efficient, painless and non-invasive transport and in vivo delivery of small molecules and/or biologically and/or pharmacologically-active agents without alteration of the agents from their current or previous forms, thereby ensuring preservation of molecular and/or agent function, minimizing toxicity while yielding optimal bioavailability for the patient as well as providing for positive patient outcomes.

### BACKGROUND OF THE INVENTION

The efficient and repeatable administration of optimal levels of both small molecule and/or biological and/or pharmacologically-active agents generally relies on the invasive intramuscular or intravenous administration routes for the molecules and/or agents by way of subcutaneous (SQ) or intramuscular (IM) injection or intravenous (IV) administration.

There are many reasons to use invasive methods (like SQ, IM and IV) to effect the administration of the molecular and/or biological and/or pharmacologically-active agents, even though such invasive methods have drawbacks. One such example is diabetes, a chronic illness that requires continuing medical care, patient self-management, and education on the part of the patient, in order to prevent acute complications as well as to reduce the risk of long-term complications Like most chronic illnesses, diabetes is difficult and complex to manage, and requires that many issues be addressed, e.g., glycemic control and a host of others. A large body of evidence exists to support a range of interventions to improve diabetes outcomes. Diabetes is a group of diseases marked by high levels of blood glucose (also called blood sugar) resulting from insulin resistance and defects in insulin production, or impaired activity of the insulin molecule. Diabetes can lead to serious complications and premature death, but people with diabetes can take steps to control the disease and lower the risk of complications. The hallmark of treatment for diabetes is the daily injection of insulin to control levels of blood sugar. The costs and impact of routine injection of medication upon a person's quality of life is substantial on many levels. A system that would allow diabetic patients to take their daily insulin, not as a syringe injection, but instead as a self-administered, painless, non-invasive nanofluidic system, would significantly decrease both the cost and impact of invasive therapies and, perhaps most importantly, remove the fear of injection and promote better compliance. If such a product were to become available and were appropriately priced, easily accessible and properly marketed to physicians and patients, patient preferences for this non-invasive delivery technology would result in a rapid increase in patient acceptance of this new, consumer-friendly product.

The concept of a non-invasive delivery system which would rely less on the user, and improve patient outcome simply by being easier to use, while reducing time and effort, would potentially change patient outcomes. By way of example but not limitation, the sudden onset of allergic reactions initiates a cascade of physiologic changes, which may culminate in anaphylactic shock. By way of further example but not limitation, the loss of adequate insulin levels in a diabetic can result in a diabetic coma. The conventional treatment these acute events is immediate treatment with the proper therapeutic agent. It is understood that by providing emergency or life-sustaining agents quickly, life may be preserved and/or permanent loss of function prevented and/or convalescence time reduced. In the foregoing examples of anaphylactic shock and diabetes discussed above, the treatment must be administered immediately and by injection. Besides the obvious aversion of most people, particularly children, to a self-administered injection, there are other potential difficulties, such as breakage of the syringe or the injectable device, expiration of the drug contents and, most commonly, unavailability because the injectable delivery system is inconvenient to carry. Moreover, in situations in which the administration of an oral medication is necessary during an acute event, such as a myocardial infarction or a seizure, the patient may not be physically able to ingest a medication, or have sufficient time between ingestion of the therapeutic agent and absorption into the blood stream, for the therapeutic agent to provide the therapeutic benefit such as reduction in or the relief of symptoms. There is a need in the art for a more convenient, less-invasive (or preferably non-invasive) painless treatment system that provides rapid or more immediate release of the therapeutic agent.

Immediate, painless, non-invasive rapid-release systems have not been successfully developed to date. A major obstacle to the development of such non-invasive rapid-release delivery systems has been that existing technologies (e.g., transdermal transport agents, inhalation-based agents, etc.) have often resulted in the conjugation of the delivery agent to the therapeutic active agent, generally forming an unintended new and distinct agent that often has new properties and a safety profile different from the original therapeutic active agent, and therefore typically do not have the intended effect.

Another major obstacle to the development of a painless, non-invasive delivery system is the uncontrolled and inconsistent delivery of pharmacologically-active agents. Non-optimal patient outcomes are often due to variability in the levels of bioavailability of the active agent. Many oral delivery systems have shown poor absorption in the gastrointestinal (GI) tract, and therefore require increased doses. As doses are increased, an increased level of associated toxicity is also typically observed. Yet another problem in the development of non-invasive delivery systems is that these systems have not been shown to be successful at providing durability for the transport of both very small and very large molecules. In fact, it has been shown that it is difficult to transport active agents larger than 1-2 kiloDaltons (kDa) across membranes.

Another drawback to the development of painless, non-invasive delivery systems in cosmetic, diagnostic and pharmaceutical products is the reliance on the degradation of mammalian membrane(s), and/or the enhancement of mammalian membrane(s) with polar solvents in order to administer active agents. The degradation of the membranes can cause severe irritation, sores, and discomfort to the patient. The use of polar solvents has been linked to these side effects as well, and also most polar solvents are highly toxic, especially when used over long periods of time, thereby allowing for excessive accumulation in patient tissues. The placement of the delivery device (patch, gel, etc.) must be relocated for subsequent administrations in order to allow previous absorption sites to heal.

Additionally, prior art non-invasive sublingual delivery systems, primarily chemical enabler-based formulations, have generally been shown to be effective only if properly administered by the patient. However, data suggests that sublingual-type formulations are frequently improperly administered. For example, the patient may fail to understand that sublingual administration requires dissolving medicine under the tongue, and that absorption in this manner is more effective for certain dosages than simply chewing and swallowing. Additionally, no known sublingual system exists for therapeutically-active molecules larger than 1 kDa.

Some painless, non-invasive formulations for the introduction of DNA into the cell of a mammal, such as those described in U.S. Pat. No. 6,624,149, do not embody the use of nanofluidic systems. Other prior art formulations, such as those described in U.S. Patent Application Publication No. 2008/0242595, have the active agent insulin covalently bonded to vitamin B₁₂. However, this conjugation of insulin to vitamin B₁₂ results in a new therapeutic active agent which is distinct from either insulin or vitamin B₁₂.

As such, a need exists for improved painless, non-invasive formulations in the field of immediate-release medicaments. More specifically, there is a need for a delivery system that may deliver an active agent in an effective amount without degradation or other loss of therapeutic activity.

New areas of science are being developed at an increasing frequency. One new area of science is the direct result of the discovery of a novel manufacturing process for the low cost, bulk manufacturing of new types of single-walled and multi-walled carbon nanotubes “CNTs” using proprietary chemistry and methodology. CNTs with lengths exceeding 10 mm have been created via Chemical Vapor Deposition (CVD) techniques. While each nanotube created by the CVD process is rooted at the substrate and terminates at the upper surface of the array, it will not be perfectly straight. As such, when placed in compression, the CNTs will buckle, or deform, in a spring-like fashion. Plasma-Enhanced Chemical Vapor Deposition (PECVD), on the other hand, can produce carbon nanotubes with near-perfect vertical alignment. A PECVD process is described in Ren et al., U.S. Pat. No. 6,863,942. Unfortunately, this technology suffers from two drawbacks that limit its use for delivery systems as described herein. The array height, or CNT length, for PECVD-created CNTs is limited to ˜50 microns, and the nanotubes which are created have a non-hollow, bamboo-like morphology. The diameter of CVD carbon nanotubes is typically limited to 1-20 nanometers, and that of PECVD carbon nanotubes is typically limited to 20-200 nm. The ability to grow and use CNTs with hollow cores of much greater size and lengths (see, for example, “Formulation of Nodulated Vapor Grown Carbon Fiber”, Jyh-Ming Ting, B. C. Lan, Carbon, Volume 38, Issue 14, 2000, pages 1917-1923) have become the foundation of a new industry that is focused on the large-scale purification of water for drinking, agriculture, sensors, diagnostic tools, etc., as well as for the painless, non-invasive delivery of drugs, diagnostic tools, and “Generally Regarded As Safe” (GRAS) molecules into humans and other mammals.

The ability to cost-effectively mass-produce reinforced CNT structures has birthed a new area of biophysics that encompasses the study of optimal nanoscale fluid flows, specifically as it relates to drug and/or active molecule in vivo delivery, as well as it relates to water purification and/or similar applications. Termed eleutherospherin nanofluidics, this area of study focuses on the optimization of nano-scale patterns, processes and methodologies, structural dimensions, fluid viscosity and dynamics and flow rates to determine (i) the most rapid delivery rate having the least side effects/negative feedbacks; (ii) the maximum efficacy as it relates to depths of administration and molecular flow and activity post-delivery; (iii) nanoneedle tip geometry as it relates to the fastest possible throughput with the least possible side effects/negative feedbacks, specifically as it relates to pain and discomfort of a patient/consumer using such a device; and (iv) manufacturing methodologies related to any of the areas discussed above.

The basic principles of the eleutherospherin nanofluidic-based technologies involve ensuring that end users will be able to use/administer the resulting consumer products easily, with valid and reproduceable results demonstrating little to no variance of drug delivery. As it relates to drug, inert filler, sterile fluid and GRAS molecule delivery, the drug, inert filler, sterile fluid or GRAS molecules are optimally delivered into the sub-dermal tissues in a controlled manner at consistent, predictable depths, thereby further increasing the patient's preference for eleutherospherin nanofluidic products. While novel in their utility, design and composition, all of the components of the eleutherospherin nanofluidic system of the present invention have been designed to maintain the GRAS designations of the agents they deliver (as given by the FDA or other relevant regulatory bodies, based upon the previous use of those agents in both regulated and unregulated cosmetic, medical, diagnostic and pharmaceutical products).

Some non-invasive solutions for the promotion of small molecules into mammalian tissues, involve the use of carbon nanotubes doped with magnetic metallic particles. When exposed to magnetic fields, the metallic particles push the carbon nanotubes into the tissues, and anything carried on or within the nanotube vehicle can then be released. This system is significantly different than the present invention, inasmuch as the use of metallic particles can result in unwanted accumulations of the particles in tissue, as well as the fact that the range of molecule sizes available for delivery is severely restricted by the internal diameter of these smaller carbon nanotubes and, as an example, cannot be used for the delivery of protein macromolecules, unless they are bound to the outside of the nanotubes. Additionally, the use of metallic particles requires that these systems undergo regulatory approval and are therefore not available for use in unregulated over-the-counter products such as cosmetics.

Some non-invasive solutions for the promotion of small molecules into mammalian tissues use highly pressurized fluid to penetrate into the sub-dermal tissues at an extremely high velocity. These systems differ from the present invention in that the present invention does not require the use of highly pressurized, high-velocity fluids to penetrate into the dermis. Such highly pressurized, high-velocity fluid systems are non-optimal for patients and consumers for a multitude of reasons, including high product component costs as well as the fact that many molecules, specifically including protein macromolecules like monoclonal antibodies, fusion proteins and certain GRAS molecules like vitamin B₁₂, are relatively fragile with respect to their molecular design and therefore present a high risk of physical damage (e.g., denaturation) when injected at high pressures and/or high velocities.

U.S. Pat. Nos. 8,361,053, 8,361,037, 8,162,901, 7,914,499, 7,806,867 and 7,481,792 all describe microsyringe systems that are different than the nanosyringe system described herein with respect to materials used, methodologies, processes and geometries, as well as the scale by which the materials are developed, which is significantly greater than the nanoscale sizes utilized by the present invention. Furthermore, the devices described in the aforementioned patents typically require the utilization of non-inert metals, like nickel and platinum (which are catalysts), in alloys to support the devices. Furthermore, the devices in the aforementioned patents can typically achieve a maximum penetration depth into the dermis of 1 millimeter or less, a depth that is significantly below the minimum depth of 4 millimeters required for the successful delivery of many molecules, including insulin.

### SUMMARY OF THE INVENTION

In accordance with the principles of the present invention, methods, compositions and systems for the painless, non-invasive delivery of pure fluids and ingredient-doped fluids into mammals are disclosed.

In one embodiment of the present invention, a painless, non-invasive delivery system for delivering an active agent comprises a plurality of hollow CNTs which are arranged in a self-supporting array, whereby the active agent is delivered through the hollow interiors of the CNTs of the array.

In another embodiment of the present invention, a painless, non-invasive delivery system for delivering an active agent via high tensile strength micro-syringes which are provided by the manufacturing of CNTs in a stable “array” against an impermeable substrate base. Using a lithographic technique to pattern a catalyst into ring shapes, having any desired inner or outer diameters, a standing tube of multiple aligned CNTs can be grown. The spaces between the CNT structures can be filled with another material to create a non-porous tube. These tubes can act as nano-syringes, each smaller than the diameter of a traditional acupuncture needle. 304 stainless steel has a modulus of ˜180 GPa. The modulus of a carbon nanotube can be as high as 1000 GPa, and therefore needles constructed therefrom, or from composites thereof, could be significantly more slender than those made from stainless steel, without being prone to buckling. For this embodiment of the invention, nano-syringes are developed from CNTs to have an interior diameter of at least 4 times greater than the width of the largest protein macromolecules (e.g., monoclonal antibodies). At this scale, the effects of Van der Waals forces are negligible, allowing for the efficient, uninterrupted flow of sterile saline solution from a storage volume placed opposite the nano-syringes against the substrate base. Upon the application of pressure by the consumer against the reservoir, the volume of saline solution, along with its drug or cosmetic molecules, quickly pours into the sub-dermal tissues at pre-programmed depths. With an outer diameter of approximately 900 to 90,000 nanometers each, the nanosyringes are thin enough to pass through the interstitial spaces between skin cells in a non-invasive fashion, thus reducing or eliminating disruption to dermal and subdermal tissue, even with multiple penetrations. Additionally, the nano-syringes are too thin to result in the activation of pain receptors under the skin's surface, since even the most sensitive of human neuron receptors cannot sense pain from a penetration having a size below 90,000 nanometers. By coupling this novel, painless, non-invasive delivery system with a targeted commercial product in its original, unaltered form, the delivery system overcomes many of the shortcomings often seen in the less effective, competing technologies such as nasal sprays, lyophilized inhalers, creams, lotions, gels and others that often bind with the active molecule, thus altering its geometry and subsequently its function, which ultimately leads to reduced efficacy and increased toxicity to the patient. In some cases, delivery of the active agent requires the use of a “driver” which is typically a relatively caustic or toxic agent, in order to enable membrane penetration.

In one preferred embodiment, an iron catalyst is deposited (using a lithographic technique) into rings on a substrate, having an inner diameter of 700 nm to 5 microns, and an outer diameter of less than 70 microns. A forest of vertically aligned carbon nanotubes are grown from the lithographically-defined catalyst, to a height of greater than 5 mm, thereby creating a tube or pipe.

In one preferred embodiment, the carbon nanotube “pipes” are overcoated with a second material to make them nonporous to fluids and thereby act as a needle. This second, overcoat material can be selected from the group of materials consisting of: metals (e.g., titanium, tungsten, molybdenum, steels, etc.), carbon, oxides (e.g., silicon dioxide, aluminum oxide, titanium dioxide, etc.), nitrides (e.g., silicon nitride, titanium nitride, etc.), carbides (e.g., boron carbide, silicon carbide, etc.) and the like.

In one preferred embodiment of the present invention, a graphitizable carbon bearing compound, such as polyacrylonitrile (PAN) is coated around the carbon nanotubes and then graphitized so as to form a carbon fiber.

In one preferred embodiment of the present invention, the substrate utilized for nanotube growth is made from a metal (e.g., titanium, stainless steel, etc.).

In one preferred embodiment of the present invention, the substrate utilized for nanotube growth may be perforated with holes that connect the inner diameter of the hollow CNT microneedles to the backside of the substrate.

In addition to the foregoing, this invention relates to delivery systems for therapeutically active agents and, in accordance with the present invention, there are provided compositions and methods useful for the in vivo delivery of pharmacologically active agents including pharmaceuticals, small-molecule biopharmaceuticals, nutraceuticals, genetic material(s), vaccines, macro-molecule biopharmaceuticals (also referred to as biologics), proteins, hormones and/or combinations thereof, in which the pharmacologically active agent is delivered painlessly and non-invasively. In particular, protein-based biopharmaceuticals, genetic material(s) and vaccines have required intravenous and/or transdermal injection for delivery, rather than oral administration, because of the degradation of proteins by digestive enzymes and low pH. Additionally, most pharmacologically-active agents generally have low bioavailability with oral or nasal delivery vs. IV, IM or SQ delivery, resulting in increased dosing, and hence a higher risk-benefit profile, than is possible with the present invention. An example of such is the osteoporosis medication Ibandronate, which requires 150 mg orally per month on an empty stomach or 3 mg by IV every 3 months. Thus, the present invention embodies a unique solution to both invasive delivery and improved bioavailability for pharmacologically active agents, sterile fluids and inert fillers currently delivered by nasal, oral, vaginal and/or rectal methods without altering them from their current or previous form.

In addition, this invention relates to inert filler delivery systems and, in accordance with the present invention, there are provided compositions and methods useful for the in vivo delivery of inert filler agents for cosmetic and reconstructive purposes. The present solution embodies a unique solution to increase visual appearance resulting from the in vivo delivery of the inert fillers as well as a painless, non-invasive method for doing so.

This invention also relates to delivery systems of Generally Regarded As Safe (GRAS) agents, and in accordance with the present invention, there are provided compositions and methods useful for the in vivo delivery of GRAS agents for cosmetic and health-related purposes. The present invention embodies a unique solution to increase visual appearance resulting from the in vivo delivery of the cosmetics as well as a painless, non-invasive method for doing so without altering them from their current or previous form.

This invention also relates to sterile fluid delivery systems and, in accordance with the present invention, there are provided compositions and methods useful for the in vivo delivery of sterile fluids for medical and cosmetic purposes. The present solution embodies a unique solution to increase visual appearance resulting from the in vivo delivery of the sterile fluids as well as a painless, non-invasive method for doing so.

In one preferred form of the present invention, there is provided apparatus for delivering an active agent to a patient, said apparatus comprising:

a carrier comprising a flexible concave member; and

a syringe mounted to said flexible concave member, said syringe comprising:
a chamber having a proximal end and a distal end and containing an active agent to be delivered to a patient, wherein said distal end of said chamber comprises a wafer substrate having a plurality of openings formed therein and having plurality of hollow fibers extending distally therefrom;
a locking mechanism disposed in telescoping relation with said chamber;
a support band disposed in telescoping relation with said locking mechanism;
one or more retention mechanisms that releasably secure said locking mechanism to said support band; and
a plunger having a proximal end and a distal end and a telescoping rod disposed therebetween, wherein said distal end comprises a plunger disc disposed in said chamber and wherein said proximal end of said plunger comprises a comfort top that communicates with said flexible concave member of said carrier; and
a spring disposed between said comfort top and said proximal end of said chamber so as to bias said comfort top proximally away from said proximal end of said chamber;

wherein when a distally-directed force is applied to said comfort top, said force overcomes said one or more retention mechanisms, moves said chamber and said locking mechanism as a unit distally and telescopically relative to said support band thereby deploying said hollow fibers distally beyond said support band and into the patient, and further wherein continued application of the distally-directed force to said plunger top overcomes the proximal bias of said spring and causes said plunger disc to move distally and push the active agent out of said chamber through said openings in said wafer substrate, through said hollow fibers and into the patient.

In another preferred form of the present invention, there is provided a method for delivering an active agent to a patient, said method comprising:

providing apparatus for delivering an active agent to a patient, said apparatus comprising:
a carrier comprising a flexible concave member; and
a syringe mounted to said flexible concave member, said syringe comprising:
   a chamber having a proximal end and a distal end and containing an active agent to be delivered to a patient, wherein said distal end of said chamber comprises a wafer substrate having a plurality of openings formed therein and having plurality of hollow fibers extending distally therefrom;
   a locking mechanism disposed in telescoping relation with said chamber;
   a support band disposed in telescoping relation with said locking mechanism;
   one or more retention mechanisms that releasably secure said locking mechanism to said support band; and
   a plunger having a proximal end and a distal end and a telescoping rod disposed therebetween, wherein said distal end comprises a plunger disc disposed in said chamber and wherein said proximal end of said plunger comprises a comfort top that communicates with said flexible concave member of said carrier; and
   a spring disposed between said comfort top and said proximal end of said chamber so as to bias said comfort top proximally away from said proximal end of said chamber;
wherein when a distally-directed force is applied to said comfort top, said force overcomes said one or more retention mechanisms, moves said chamber and said locking mechanism as a unit distally and telescopically relative to said support band thereby deploying said hollow fibers distally beyond said support band and into the patient, and further wherein continued application of the distally-directed force to said plunger top overcomes the proximal bias of said spring and causes said plunger disc to move distally and push the active agent out of said chamber through said openings in said wafer substrate, through said hollow fibers and into the patient;

disposing said carrier against the skin of the patient at a desired location;

applying a distally-directed force to said comfort top to overcome said one or more retention mechanisms, whereby to move said chamber and said locking mechanism as a unit distally and telescopically relative to said support band thereby deploying said hollow fibers into the skin of the patient;

continuing the application of said distally-directed force to move said plunger disc distally so as to push the active agent out of said chamber through said openings in said wafer substrate, through said hollow fibers and into the patient.

In another preferred form of the present invention, there is provided apparatus for delivering an active agent to a patient, said apparatus comprising:

a flexible carrier comprising a concavity; and

a syringe mounted within said concavity of said flexible carrier, said syringe comprising:
a hollow base comprising a distal end and a proximal end;
a cap movably mounted to said proximal end of said hollow base;
a spring disposed between said distal end of said hollow base and said cap, said spring being configured to proximally bias said cap;
a wafer substrate comprising a distal surface and a proximal surface, and a plurality of openings extending between said distal surface and said proximal surface, said wafer substrate being movably disposed within said hollow base;
a plurality of nano-needles extending distally from said distal surface of said wafer substrate, wherein each of said nano-needles comprises a distal end and a proximal end, and a lumen extending therebetween, and further wherein said each lumen of each of said plurality of nano-needles is aligned with said openings formed in said wafer substrate;
a rod having a distal end and a proximal end, wherein said distal end of said rod is mounted to, and extends between, said wafer substrate and said cap;
a needle support plate mounted to the distal end of said hollow base, said needle support plate comprising a plurality of openings sized to receive a plurality of nano-needles therein;
a timing ring having a distal end and a proximal end, and a plunger disc mounted to said distal end of said timing ring, wherein said timing ring and said plunger disc are disposed coaxially about said rod intermediate said cap and said wafer substrate, whereby to define a chamber between said plunger disc and said wafer substrate for containing the active agent which is to be delivered to a patient, and further wherein said timing ring is configured to selectively move longitudinally relative to said rod and is configured to selectively rotate relative to said rod;
a torsional spring disposed between said cap and said distal end of said timing ring;
a locking ring mounted to said timing ring and to said hollow base, said locking ring being configured to selectively permit said timing ring to rotate relative to said rod;

wherein when said cap is moved distally, (i) said wafer substrate moves distally so as to project said plurality of nano-needles through said openings formed in said needle support plate and into the patient, and (ii) said locking ring releases said timing ring, whereby to allow said torsional spring to bias said timing ring and said plunger disc distally and thereby force the active agent contained in said chamber into said openings formed in said substrate, through said lumens of said plurality of nano-needles, and into the patient.

In another preferred form of the present invention, there is provided a method for delivering an active agent to a patient, said method comprising:

providing apparatus for delivering an active agent to a patient, said apparatus comprising:
a flexible carrier comprising a concavity; and
a syringe mounted within said concavity of said flexible carrier, said syringe comprising:
   a hollow base comprising a distal end and a proximal end;
   a cap movably mounted to said proximal end of said hollow base;
   a spring disposed between said distal end of said hollow base and said cap, said spring being configured to proximally bias said cap;
   a wafer substrate comprising a distal surface and a proximal surface, and a plurality of openings extending between said distal surface and said proximal surface, said wafer substrate being movably disposed within said hollow base;
   a plurality of nano-needles extending distally from said distal surface of said wafer substrate, wherein each of said nano-needles comprises a distal end and a proximal end, and a lumen extending therebetween, and further wherein said each lumen of each of said plurality of nano-needles is aligned with said openings formed in said wafer substrate;
   a rod having a distal end and a proximal end, wherein said distal end of said rod is mounted to, and extends between, said wafer substrate and said cap;
   a needle support plate mounted to the distal end of said hollow base, said needle support plate comprising a plurality of openings sized to receive a plurality of nano-needles therein;
   a timing ring having a distal end and a proximal end, and a plunger disc mounted to said distal end of said timing ring, wherein said timing ring and said plunger disc are disposed coaxially about said rod intermediate said cap and said wafer substrate, whereby to define a chamber between said plunger disc and said wafer substrate for containing the active agent which is to be delivered to a patient, and further wherein said timing ring is configured to selectively move longitudinally relative to said rod and is configured to selectively rotate relative to said rod;
   a torsional spring disposed between said cap and said distal end of said timing ring;
   a locking ring mounted to said timing ring and to said hollow base, said locking ring being configured to selectively permit said timing ring to rotate relative to said rod;

wherein when said cap is moved distally, (i) said wafer substrate moves distally so as to project said plurality of nano-needles through said openings formed in said needle support plate and into the patient, and (ii) said locking ring releases said timing ring, whereby to allow said torsional spring to bias said timing ring and said plunger disc distally and thereby force the active agent contained in said chamber into said openings formed in said substrate, through said lumens of said plurality of nano-needles, and into the patient;

disposing said flexible carrier against the skin of the patient at a desired location;

applying a distal force so as to move said cap distally, whereby to (i) advance said plurality of nano-needles into the skin of the patient, and (ii) force the active agent contained in said chamber into said openings formed in said substrate, through said lumens of said plurality of nano-needles, and into the patient; and

allowing said cap to move proximally under the power of said spring disposed between said distal end of said hollow base and said cap, whereby to move said wafer substrate and said plurality of nano-needles proximally, whereby to withdraw said nano-needles from the skin of the patient.

In another preferred form of the present invention, there is provided a nano-needle comprising a plurality of carbon nanotubes having a matrix material filling the interstitial spaces between said carbon nanotubes.

In another preferred form of the present invention, there is provided a method for making a nano-needle comprising a plurality of carbon nanotubes, said method comprising:

providing a wafer substrate having one or more openings extending therethrough;

depositing a catalyst around the periphery of said one or more openings extending through said wafer substrate;

activating said catalyst so that said catalyst forms islands around the periphery of said one or more openings;

growing a plurality of carbon nanotubes from said islands;

applying a matrix material to the interstitial spaces between said carbon nanotubes so as to form a hollow nano-needle having a diameter that is roughly defined by the periphery of said one or more openings.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1-3 are schematic views of a novel nano-syringe system formed in accordance with the present invention;

FIGS. 4-6 are schematic views showing further details of the novel nano-syringe system shown in FIGS. 1-3;

FIG. 6A is a schematic view of the syringe component of the novel nano-syringe system shown in FIGS. 4-6, shown in its starting condition;

FIG. 6B is a schematic view of the syringe component of the novel nano-syringe system shown in FIGS. 4-6, shown in its intermediate condition;

FIG. 6C is a schematic view of the syringe component of the novel nano-syringe system shown in FIGS. 4-6, shown in its final condition;

FIG. 7 is a schematic view showing CNTs adhered to a substrate formed in accordance with the present invention;

FIG. 8 is a photograph of exemplary CNTs formed in accordance with the present invention;

FIG. 9 is schematic view of single-walled CNTs formed in accordance with the present invention;

FIG. 10 is a schematic view of multi-walled CNTs formed in accordance with the present invention;

FIG. 11 is a schematic view of the CNTs of the present invention interacting with dermal tissue;

FIGS. 12A-12E are schematic views of a novel nano-needle formed in accordance with the present invention, wherein the novel nano-needle comprises a plurality of CNTs extending out of a wafer substrate and arranged so as to collectively form a hollow tubular meta-structure;

FIG. 13 is a photograph of an aligned array of CNTs at low magnification;

FIG. 14 is a photograph of the novel nano-needles of FIGS. 12A-12E after a matrix material has been deposited within the interstitial spaces between CNTs;

FIGS. 15-19 are schematic views showing another novel nano-syringe system of the present invention which utilizes the novel nano-needles of FIGS. 12A-12E, 13 and 14, combined with a modified form of deployment apparatus;

FIGS. 20-22 are schematic views showing the novel deployment apparatus of the novel nano-syringe system of FIGS. 15-19;

FIGS. 23 and 24 are schematic views showing further details of the novel deployment apparatus of FIGS. 20-22;

FIGS. 25-33 show the novel deployment apparatus of FIGS. 20-22, 23 and 24 used to deploy the novel nano-needle of FIGS. 12A-12E, 13 and 14;

FIGS. 33A and 33B show another novel deployment apparatus for use with the novel nano-syringe system of FIGS. 15-19;

FIGS. 34A-34E show a novel process for forming alternative tubular structures for use with the present invention; and

FIGS. 35 and 36 show an exemplary tungsten tubular structure, formed in accordance with the process depicted in FIGS. 34A-34E, extending out of the skin of a patient.

### DETAILED DESCRIPTION OF PREFERRED EMBODIMENTS

The present invention discloses painless, non-invasive delivery systems and methods that provide an effective administration to mammals of one or more active pharmacological ingredients (i.e., an “active agent”) through cellular membranes.

### DEFINITIONS

As used herein, the term “membrane” refers to a selective cellular barrier that is selectively permeable and controls the movement of substances into and out of cells. Membranes are generally comprised of proteins and lipids. Additionally, membranes include a cell potential (i.e., electrical charge). The term “membranes” is intended to encompass all cellular membranes, preferably animal, and more preferably mammalian, and most preferably human. The membrane can be, without limitation, both connective and epithelial membranes. An example of a connective membrane includes a synovial membrane. Examples of epithelial membranes include the skin, mucosal membranes and serous membranes. The membranes can be dry membranes or wet membranes.

Examples of additional mammalian membranes, for both humans and other mammals, include mesenteric, dermal, epidermal, blood-brain barrier, intervaginal, rectal, colonic, ocular, internasal and tympanic membranes.

Embodiments of the present invention may provide for the delivery of physiologically-active agents to the bloodstream without passing through the gastrointestinal (GI) tract. Preferred embodiments allow the active ingredient to enter specific organs, such as the skin for dermal application. In certain embodiments, the delivery system provides targeted delivery of the active agent. For example, the blood supply will directly receive the active agent, without requiring the active agent to pass through the liver via the GI tract. Elimination of the “first-pass” detoxification allows for a decreased burden to the liver, as well as improving the onset of the action of the active “drug” and typically reducing the required delivery dose.

Embodiments of the present invention are intended for administration to animals in need, particularly mammals, and more particularly humans. Accordingly, for purposes herein, the terms “patient”, “consumer” or “subject” refer to an individual who is in need of an active agent for therapeutic, prophylactic, cosmetic or preventative reasons, either as an unregulated over-the-counter product or as a regulated product that is prescribed by a medical or clinical professional. A patient can refer to a mammal, including, but not limited to, humans, monkeys, rats, cows, sheep, dogs, cats, goats, etc. A patient may refer to an adult or a child.

For the purposes herein, the term “active agent” refers to any molecule administered for a therapeutic benefit. An active agent can comprise a pharmacologically-active agent, a sterile fluid and/or an inert filler substance. An active agent can be physiologically active. An active agent may refer to, without limitation, pharmaceuticals, including large-molecule pharmaceuticals, small molecule pharmaceuticals, biopharmaceuticals (also referred to as “biologics”), large molecule biopharmaceuticals, small molecule biopharmaceuticals, nutraceuticals, genetic material (including DNA and RNA), preferably isolated or purified, recombinant nucleic acid vectors (e.g., as plasmids, cosmids, etc.), vaccines, proteins, peptides, hormones, organic or inorganic molecules, nanoparticles (e.g., nanocarbons, nanodiamonds, silicons, sulfates/sulfites technologies, etc.) or any combination thereof. Examples of proteins/peptides may include antibodies (e.g., monoclonal antibodies), glycosylated and non glycosylated molecules, fusion proteins, protein fragments, sterols and bioidentical compounds, as well as a wide variety of combinations of amino acids and hormones, both human and plant-based.

Additional examples of suitable active agents are provided herein. One or more active agents may be used. A combination of active agents may be provided, concurrently or serially, preferably in any order. The size of the active agent may range from between about less than 1 kDa to about 500 kDa or more. One skilled in the art may prefer to use such a formulation for the non-invasive delivery of active agents, where the size of the active agents range from about less than 1 kDa to about 20 kDa, while other skilled artisans prefer to use the formulation with active agents from about 2 kDa to about 200 kDa, or from about 200 kDa to about 500 kDa or even larger. In some embodiments, the active ingredient may range up to about 1000 kDa.

For the purposes herein, an “unaltered” therapeutic active agent refers to an unchanged or unaltered active agent when transported. An unchanged therapeutic active agent is an active agent that undergoes no molecular or irreversible changes. Preferably, the active agent does not undergo any irreversible changes in chemical structure, properties, or activity. Even more preferably, the active agents in the present disclosure are transported across membranes in their original state. In one preferred embodiment the system maintains, or at least does not alter, the pharmaceutical and/or therapeutic effect of the active agents, so that the delivery system provides the patient with the expected and appropriate effect of the active agent. For example, an unaltered active agent is not glycosylated solely for the purposes of absorption (wherein the therapeutic agent may not be glycosylated). As another example, the unaltered active agent does not become conjugated to another molecule for the purposes of absorption (wherein the therapeutic active agent is not normally conjugated to another molecule). As still another example, an unaltered active agent is not cleaved into a smaller portion or fragment in order for successful absorption (i.e., absorption occurs with the active agent intact).

In preferred embodiments of the present invention, the vehicles of the system and the active agent are not covalently bonded or altered in any way beyond their original formulation. In the most preferred embodiments, the active agent and non-invasive system are bound together through ionic interactions. Interactions of the active agent with the non-invasive delivery system do not require, and preferably avoid, conjugation, or the formation of new moieties, that may reduce the therapeutic effectiveness of the active agents. It is preferable that the system does not change the stoichiometry or physiologic functions of the treating agent, and thus, the physiologic therapeutic effect of the active agent is preserved.

The term “an effective amount,” as used herein, is encompassed by the desired dosage amounts and dose frequency schedule, particularly when coupled with prevention, treatment, or management of one or more conditions requiring therapeutic treatment. Effective amounts may vary by subject, disease and active agent, but are generally known in the art or are determinable or optimizable by routine testing.

For the purposes herein, “prophylaxis” may refer to the prevention of the symptoms of a disease, a delay in onset of the symptoms of a disease, or a lessening in the severity of subsequently developed disease symptoms. The terms “prevent”, “preventing” and “prevention” refer herein to the inhibition of the development or onset of a disorder or the prevention of the recurrence, onset or development of one or more symptoms of a disorder in a subject resulting from the administration of a therapy (e.g., a prophylactic or therapeutic agent), or the administration of a combination of therapies (e.g., a combination of prophylactic or therapeutic agents).

For the purposes herein, “therapy” or “treatment” or “treat” can mean a complete abolishment of the symptoms of a disease or a decrease in the severity of the symptoms of the disease. For the purposes herein, the term “substantially” is intended to include variations from the absolute condition, e.g., about 90 percent, preferably about 95 percent, more preferably about 99 percent of the absolute condition. In preferred embodiments, the term “substantially” refers to 99.9 percent or even 99.99 percent of the absolute.

For the purposes herein, the term “immediate environment” refers to the area at, or substantially near, where the formulation is directly administered. Preferably, the immediate environment is directly at the site of administration where the formulation is administered. Preferably, the immediate environment includes both the formulation and the site of administration where the formulation is administered and may be in contact with the patient/consumer. The immediate environment may allow for the formulation to interact directly with the membrane to allow the system to facilitate absorption. In some embodiments, the immediate environment is within about 0-5 cm or less of the administration site.

### Nano-Syringe Comprising a Plurality of Hollow Fibers

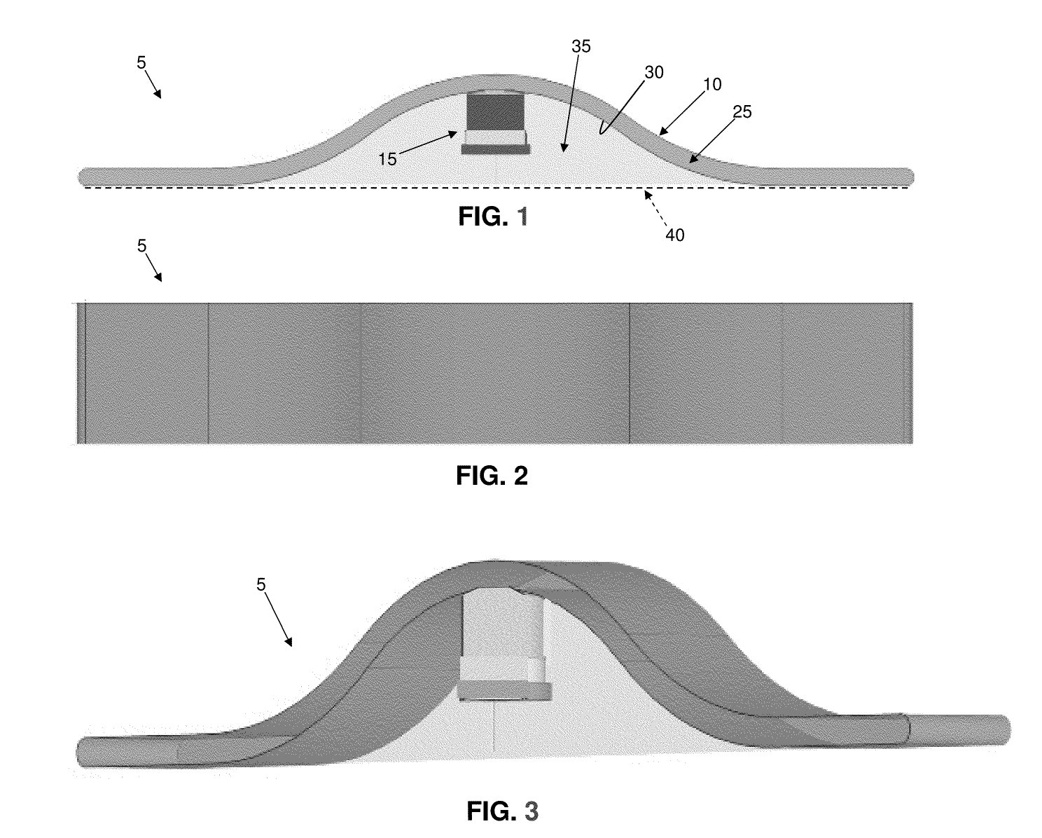

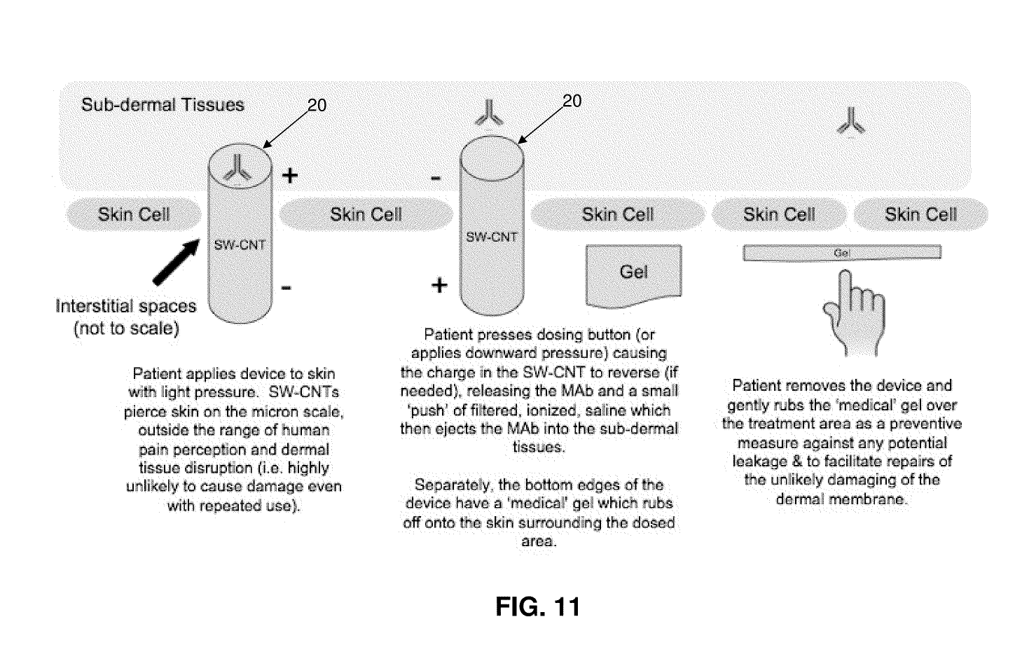

In one form of the invention, and looking now at FIGS. 1-11, there is provided a nano-syringe system 5 comprising a carrier 10 having a syringe 15 carried thereby, wherein syringe 15 comprises a plurality of hollow fibers 20.

More particularly, carrier 10 comprises a flexible concave member 25 having syringe 15 mounted within its concavity 30. The remainder of the volume of concavity 30 is filled with a gel 35. Preferably, a peel-away strip 40 covers the bottom surface of flexible concave member 25, sealing syringe 15 and gel 35 until the time of use.

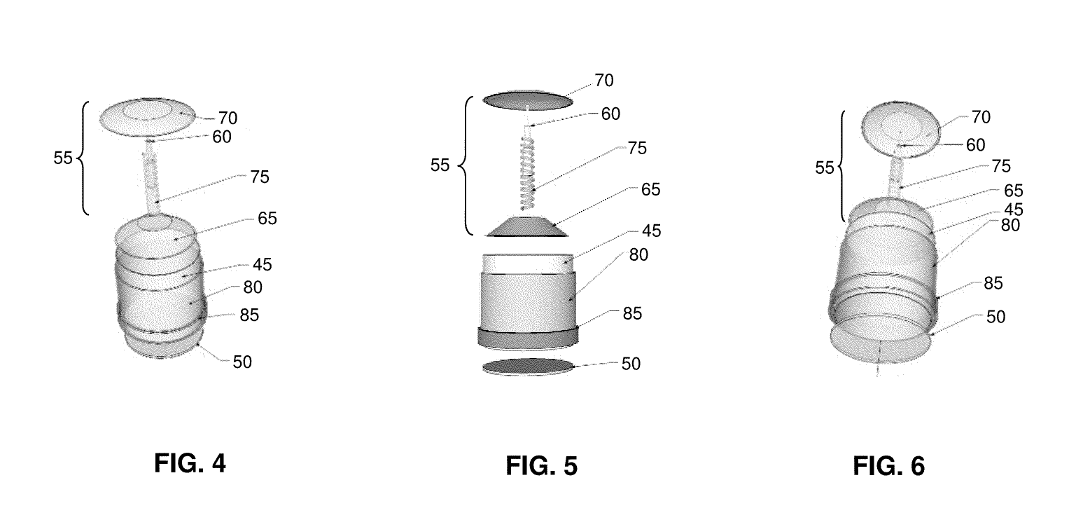

Syringe 15 is shown in further detail in FIGS. 4-6, 6A, 6B, 6C, 7 and 8. Syringe 15 comprises a chamber 45 having a wafer substrate 50 closing off the distal end of chamber 45. Wafer substrate 50 supports hollow fibers 20, with hollow fibers 20 being in fluid communication with chamber 45, as will hereinafter be discussed. The distal end of a plunger 55 is movably disposed within chamber 45. More particularly, plunger 55 comprises a telescoping plunger arm 60 having a silicone plunger disc 65 set at its distal end, and a comfort top 70 disposed at its proximal end. If desired, a spring 75 may be disposed on telescoping plunger arm 60 between silicone plunger disc 65 and comfort top 70. Chamber 45 is disposed in telescoping relation with a locking mechanism 80, which is disposed in telescoping relation with a support band 85. Shear tabs 87 may be disposed between locking mechanism 80 and support band 85. As a result of this construction, when comfort top 70 is advanced distally relative to chamber 45, silicone plunger disc 65 moves distally so as to force the contents of chamber 45 to move distally, out hollow fibers 20 as will hereinafter be discussed. Locking mechanism 80, interacting with support band 85, prevents plunger 55 from activating prematurely as will also hereinafter be discussed.

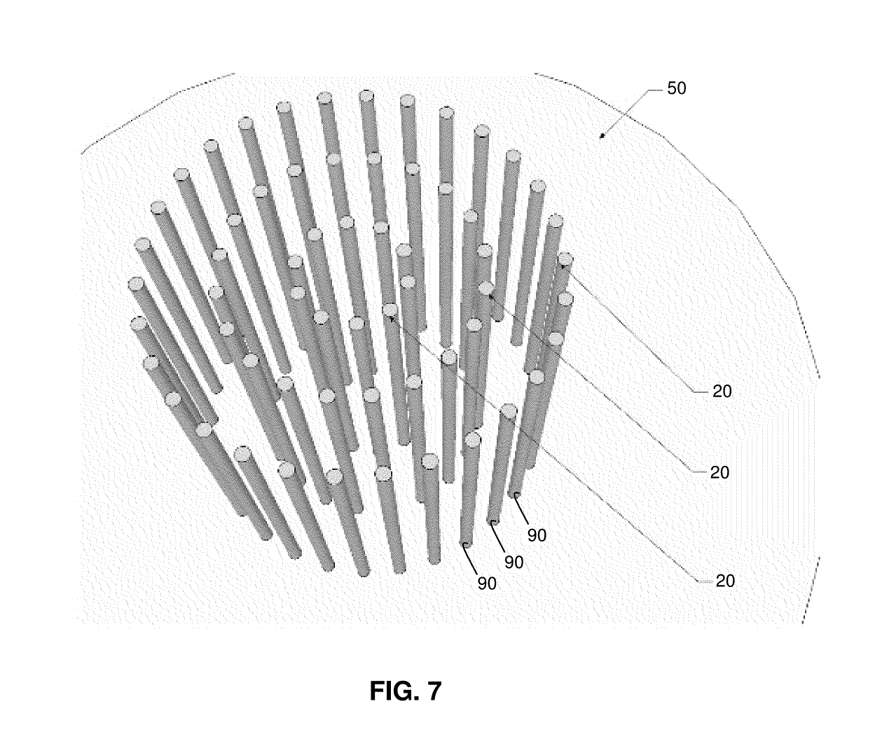

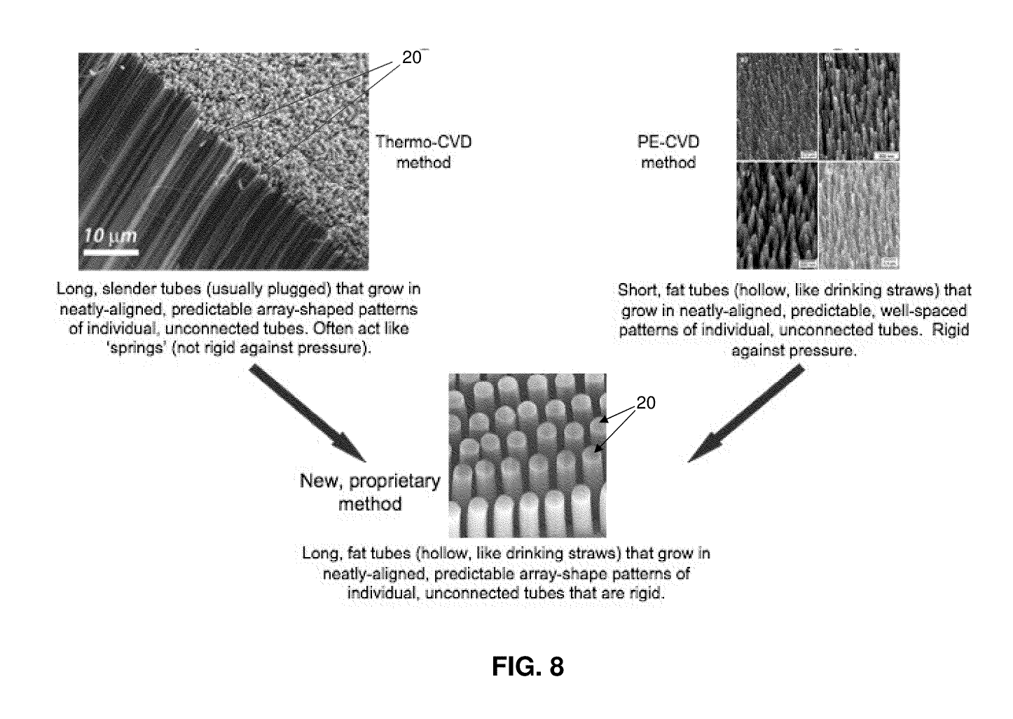

Looking now at FIGS. 7 and 8, an array of hollow fibers 20 extend distally from wafer substrate 50. As noted above, hollow fibers 20 are in fluid communication with chamber 45, e.g., via openings 90 extending through wafer substrate 50 and communicating with the interior of hollow fibers 20. A large number of hollow fibers 20 are provided, with the hollow fibers being in closely-spaced relation to one another, so that they create a self-supporting meta-structure of long, hollow tubes, each of which is capable of delivering fluid from chamber 45 to the sub-dermal tissues of a patient.

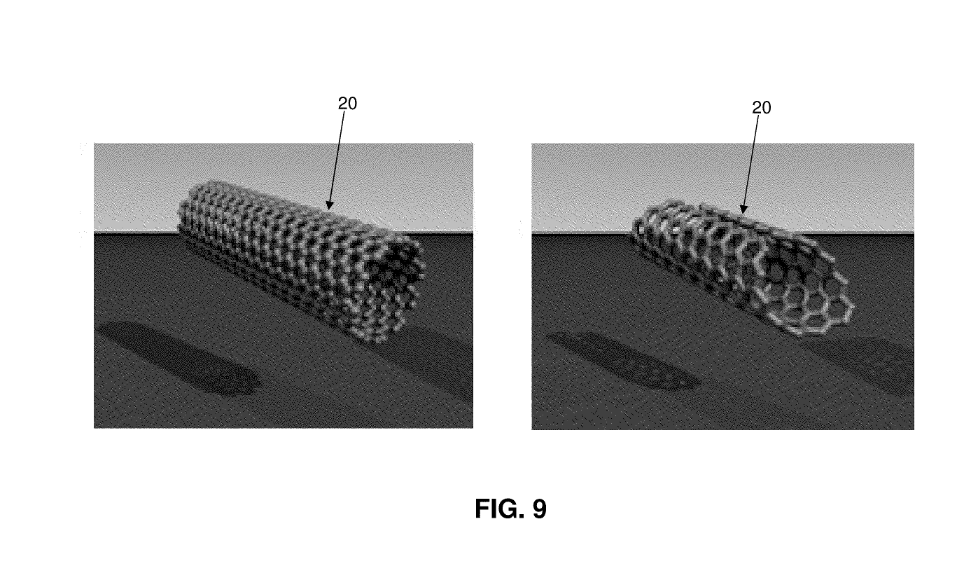

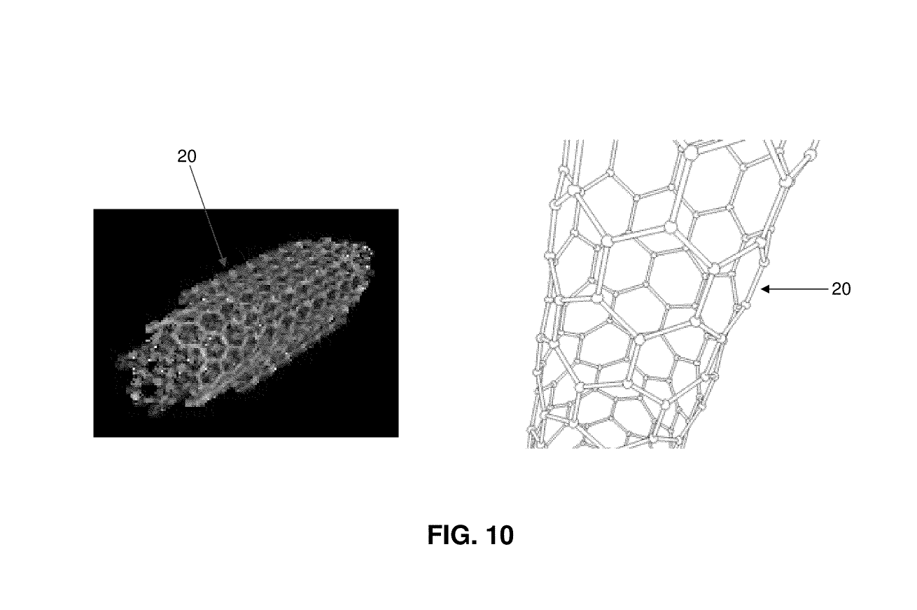

Hollow fibers may be any hollow nanostructured material, but carbon nanotubes (CNTs) 20 are one preferred material. Carbon nanotubes (CNTs) 20 may be single-walled CNTs (FIG. 9) or multi-walled CNTs (FIG. 10). Such single-walled CNTs and multi-walled CNTs are well known in the art of carbon nanotubes.

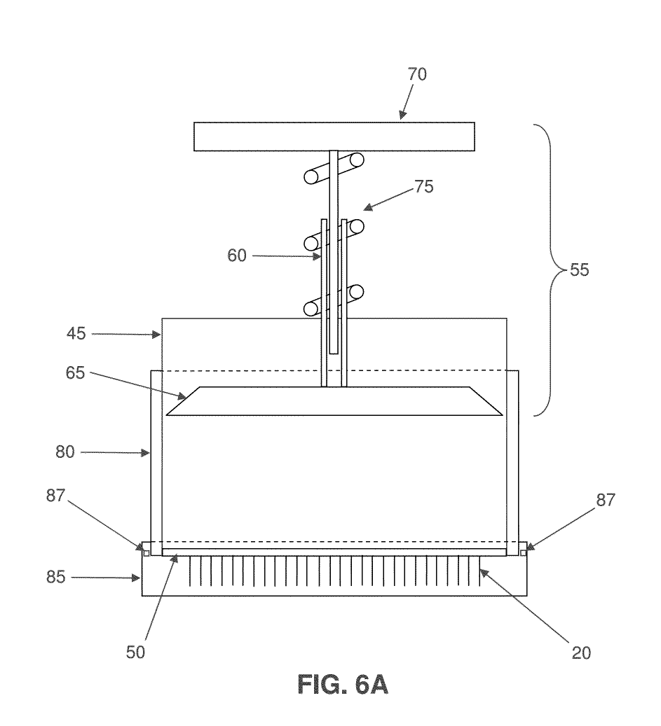

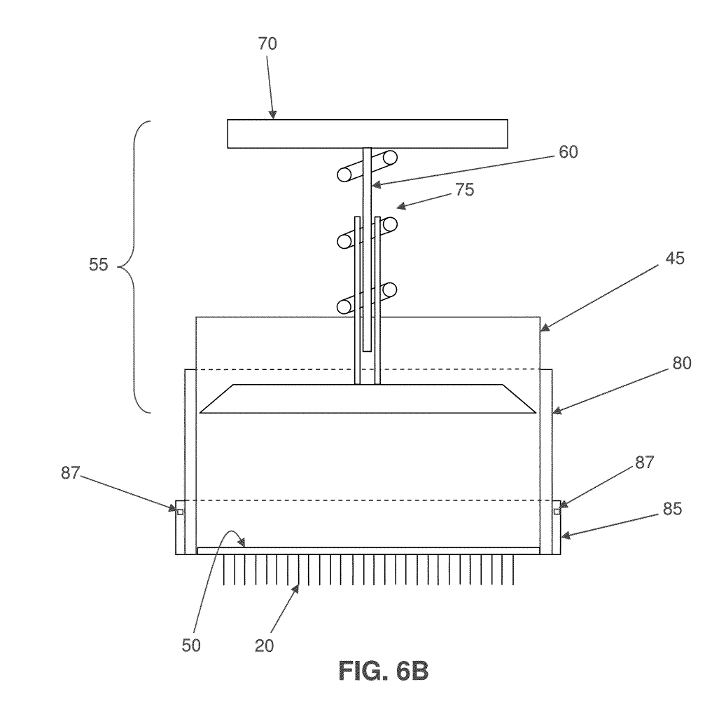

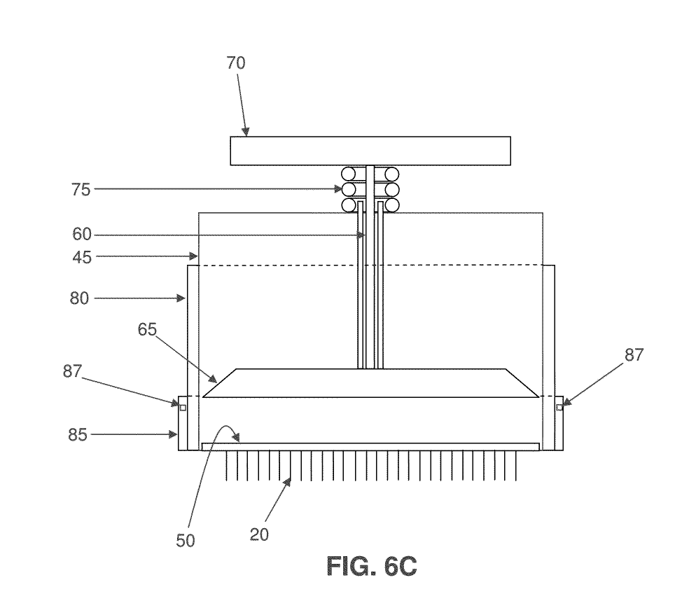

With this form of the present invention, at the time of use, nano-syringe system 5 has its peel-away strip 40 removed from the bottom surface of flexible concave member 25 of nano-syringe system 5, whereby to expose syringe 15 and gel 35. The bottom side of nano-syringe system 5 is placed against the skin of a patient at the active agent delivery site, and then the top surface of carrier 10 is depressed toward the skin of the patient. Looking now at FIGS. 6A, 6B and 6C, this action causes comfort top 70 to move distally, which causes chamber 45 to move distally, until support band 85 contacts the patient's skin. Continued distal movement of comfort top 70 causes shear tabs 87 to break, whereupon chamber 45 moves distally and inserts hollow fibers 20 into the patient's skin. With further distal movement of chamber 45 being prevented by engagement with the skin, continued distal movement of comfort top 70 overcomes the proximally-biased force of spring 75, moving silicone plunger disc 65 distally. This action forces active agent in chamber 45 out of chamber 45, through openings 90 and through hollow fibers 20 so as to deliver the contents of chamber 45 into the sub-dermal tissues of the patient. See FIG. 6C. Thereafter, nano-syringe system 5 may be removed from the patient.

### Nano-Needle Comprising a Plurality of Nanofibers (e.g., CNTs) Arranged to Form a Hollow Tubular Meta-Structure

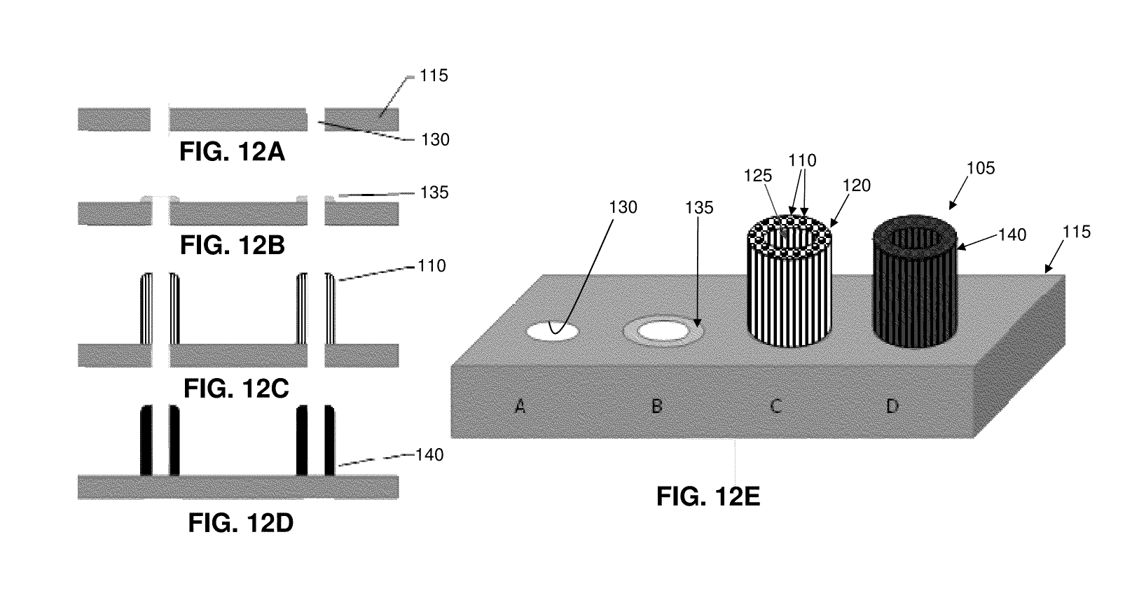

In another form of the invention, and looking now at FIGS. 12-14, there is provided a nano-needle 105 comprising a plurality of nanofibers (e.g., CNTs) 110 extending out of a wafer substrate 115 and arranged so as to collectively form a hollow tubular meta-structure 120 having a lumen 125 defined thereby, with hollow tubular meta-structure 120 thereafter being sealed as will hereinafter be discussed so as to form nano-needle 105. In this form of the invention, wafer substrate 115 comprises at least one opening 130 extending therethrough, so as to allow lumen 125 of nano-needle 105 to communicate with the active agent which is to be delivered, such that the active agent which is to be delivered flows through lumen 125 of nano-needle 105.

Thus, by replacing wafer substrate 50 and hollow fibers 20 of nano-syringe system 5 with wafer substrate 115 and a plurality of nano-needles 105, the active agent may be delivered to the sub-dermal tissue of the patient.

More particularly, FIGS. 12A-12E show an approach for manufacturing nano-needle 105.

FIG. 12A shows the wafer substrate 115 that is perforated by one or more openings 130.

FIG. 12B shows a ring of catalyst 135 deposited around the periphery of one or more openings 130. Catalyst 135 (e.g., iron, cobalt, nickel and/or another metal well known in the art of growing carbon nanotubes) is typically deposited via sputtering or evaporation techniques, and patterned using optical or electron beam lithography techniques. Multi-layer catalysts or adhesion promoting layers can also be used in catalyst ring 135 without departing from the scope of the present invention. In one preferred form of the invention, aluminum oxide is deposited atop the wafer substrate 115, before the catalytic layer is deposited, so as to promote adhesion.

FIG. 12C shows an array of CNTs 110 having been grown from catalytic ring 135. During the heating process that precedes carbon nanotube growth, the catalyst metal film, which is typically thin (e.g., approximately 1 nm) will “break up” into nanoscale islands. Each island then nucleates the growth of a carbon nanotube. A carbon nanotube will grow in a random direction until it encounters another growing carbon nanotube, at which point the carbon nanotubes may either become entangled with one another, or adhere to one another, and then grow as a pair or as a group. This tends to promote vertical alignment in the array of carbon nanotubes. In this way, the hollow tubular meta-structure 120, having a lumen 125 defined thereby, is grown out of wafer substrate 115, wherein lumen 125 of hollow tubular meta-structure 120 is aligned with the opening 130 extending through wafer substrate 115.

In FIG. 12D, a matrix material 140 is deposited within the interstitial spaces between CNTs 110 so as to form a rigid, non-porous hollow nano-needle 105 having an inner and outer diameter that is roughly defined by catalyst ring 135, and a length that is defined by the height of the nanotube array, which is governed by process conditions and growth time. The deposition of a matrix material in the interstitial spaces between the nanotubes is discussed in Nicholas: “Electrical device fabrication from nanotube formations,” US 20100140591 A1. This filing discusses the use of chemical vapor deposition and atomic layer deposition to embed and encapsulate the nanotubes completely, and references Gordon et al., “ALD of High-k dielectrics on suspended functionalized SWNTs, Electrochemical and Solid-State Letters,” 8 (4) G89-G91 (2005) and Lu et al., “DNA Functionalization of Carbon Nanotubes for Ultra-Thin Atomic Layer Deposition of High k Dielectrics for nanotube Transistors with 60 mV/decade Switching,” arXiv:cond-mat/0602454; and Fahlman et al., “CVD of Conformal Alumina Thin Films via Hydrolysis of AlH₃(NMe₂Et),” Adv. Mater. Opt. Electron 10, 135-144 (2000).

See FIG. 12E, which provides an isometric, sequential view of the aforementioned four-step process for producing nano-needle 105.

Note that in this form of the invention, the individual CNTs 110 may be substantially hollow, substantially solid or a combination thereof.

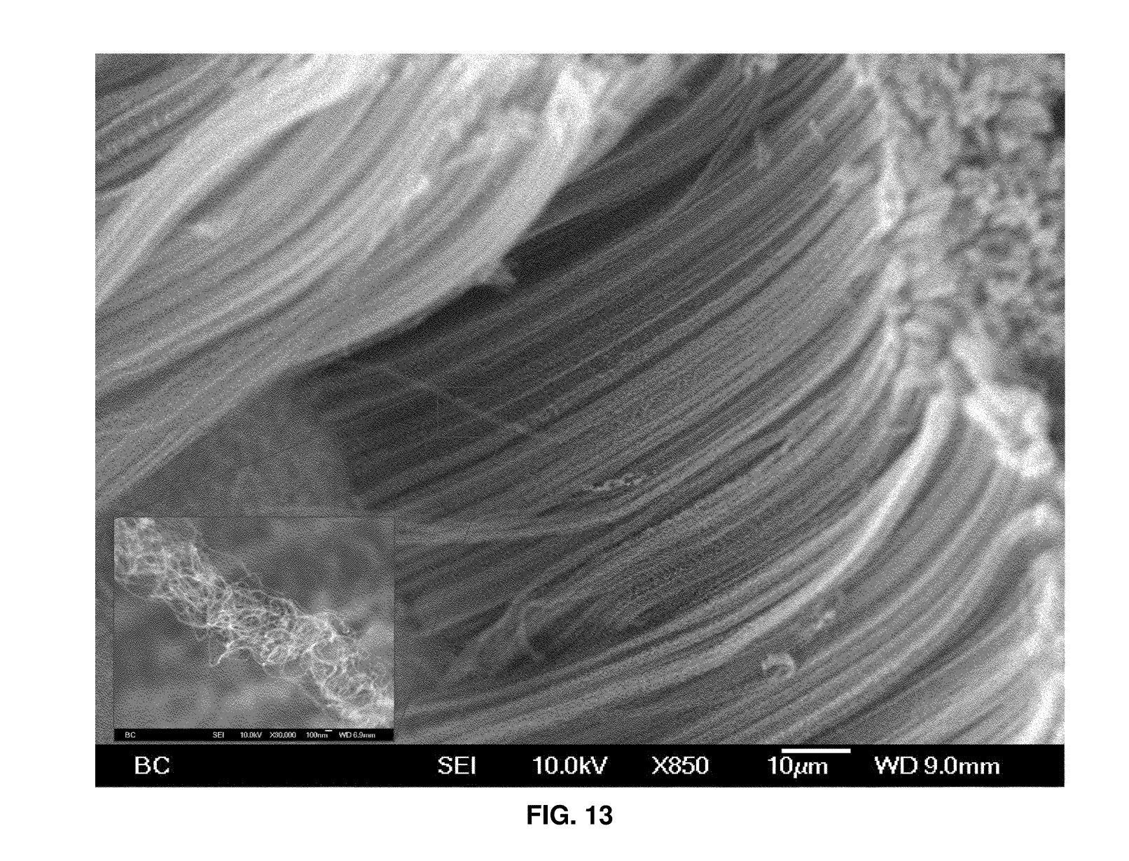

FIG. 13 shows an aligned array of CNTs 110 at low magnification. In the inset of FIG. 13, a cluster of CNTs 110 is shown, having overall parallel alignment despite significant directional wander of the constituent CNTs.

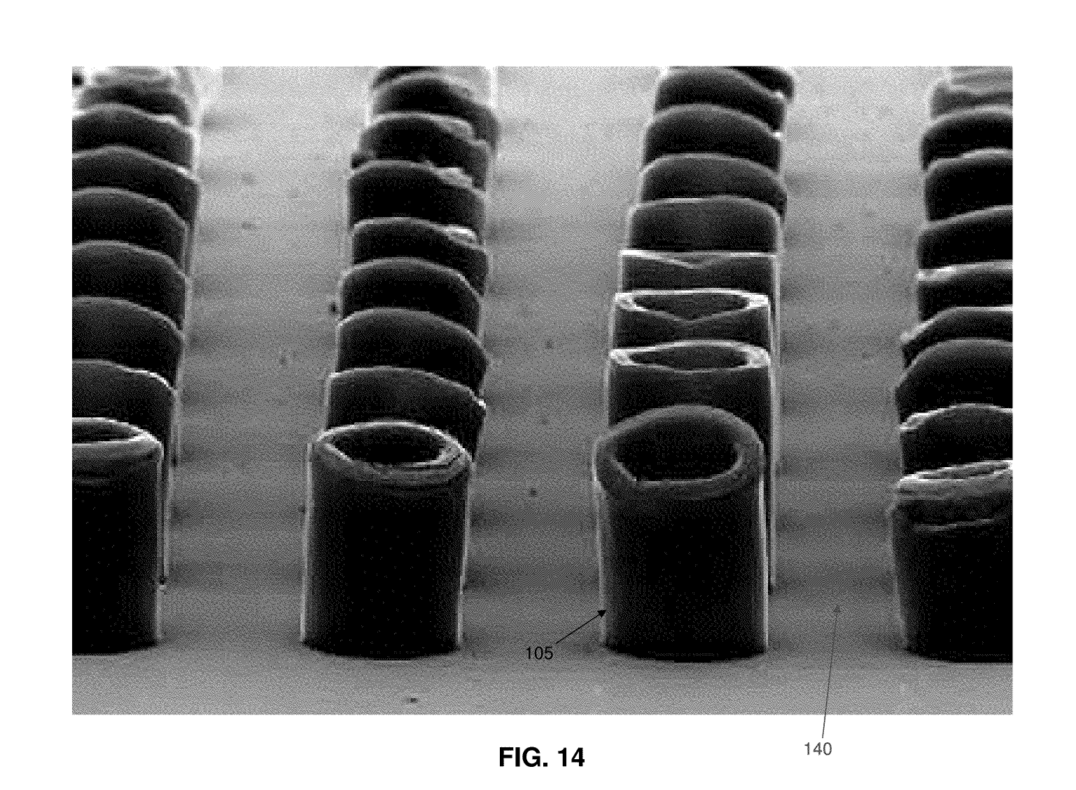

FIG. 14 shows nano-needle 105 after a matrix material 140 has been deposited within the interstitial spaces between CNTs 110.

### PREFERRED EMBODIMENT OF THE PRESENT INVENTION

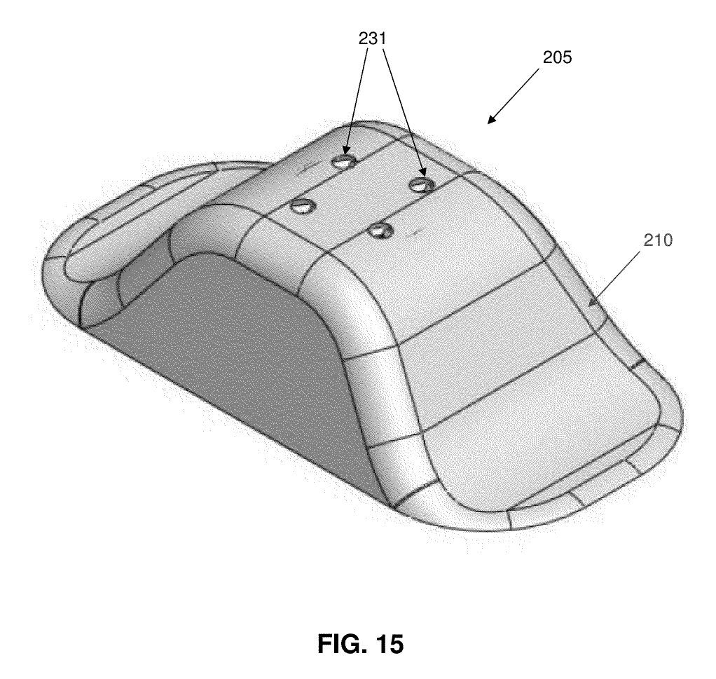

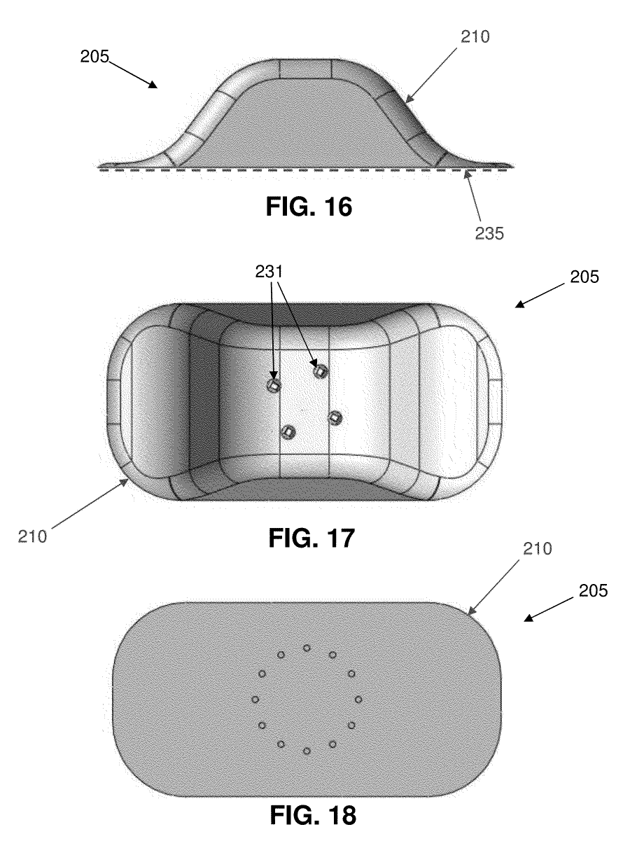

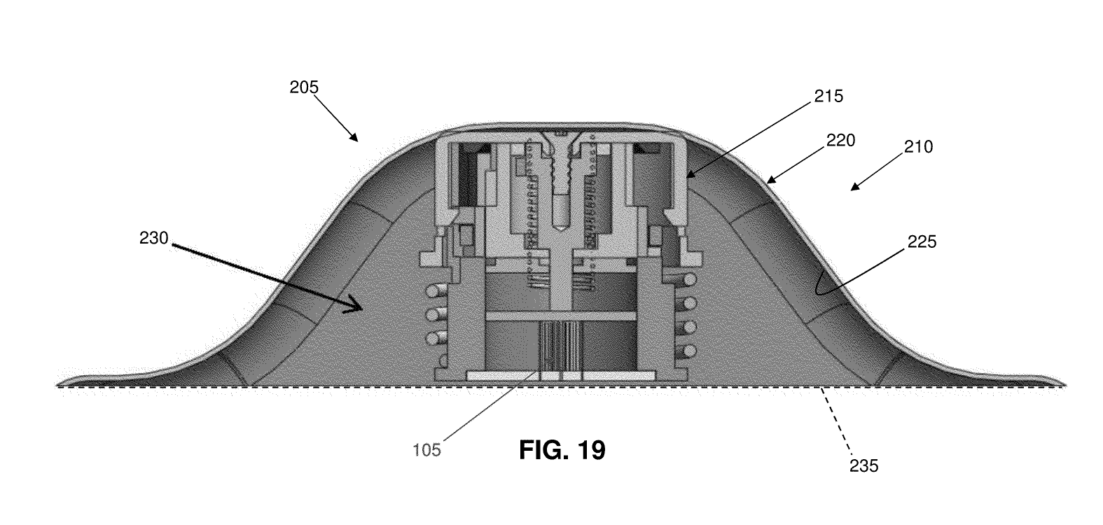

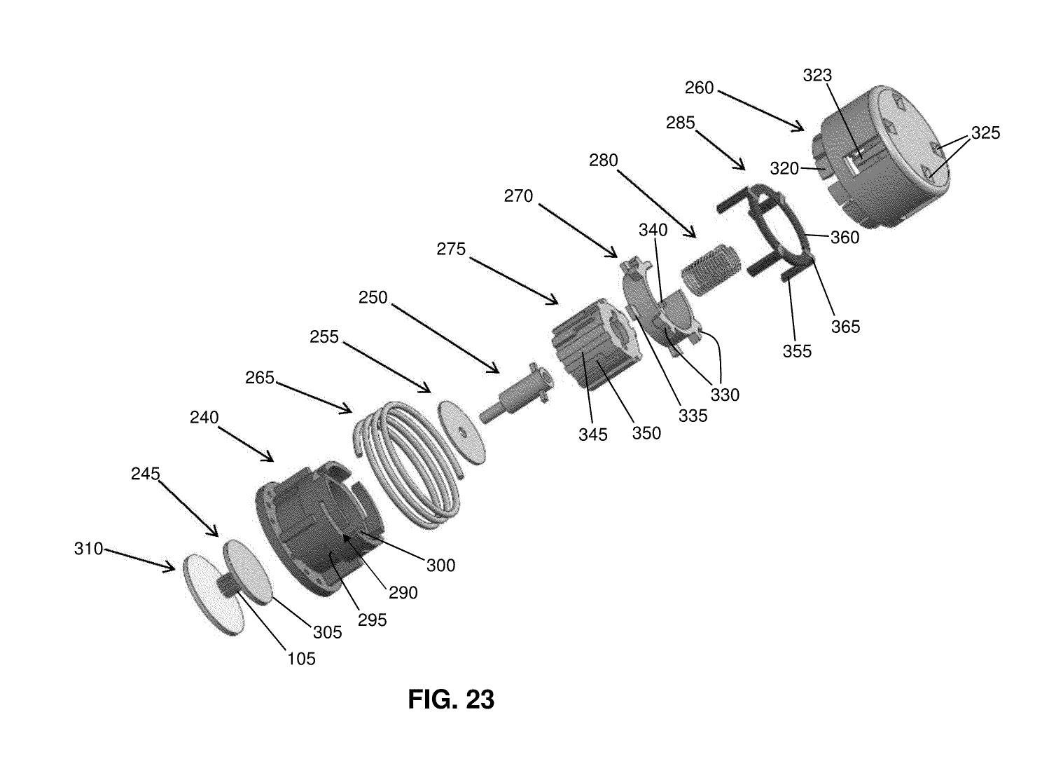

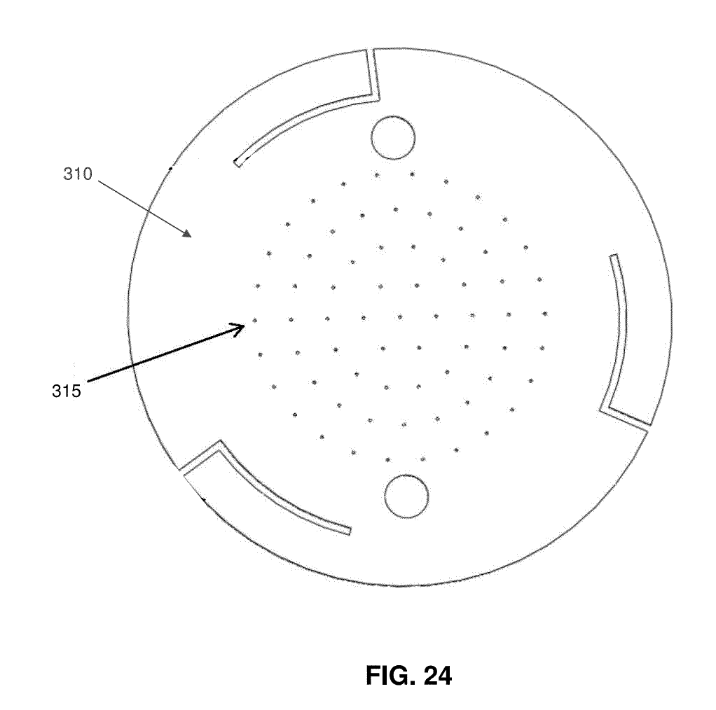

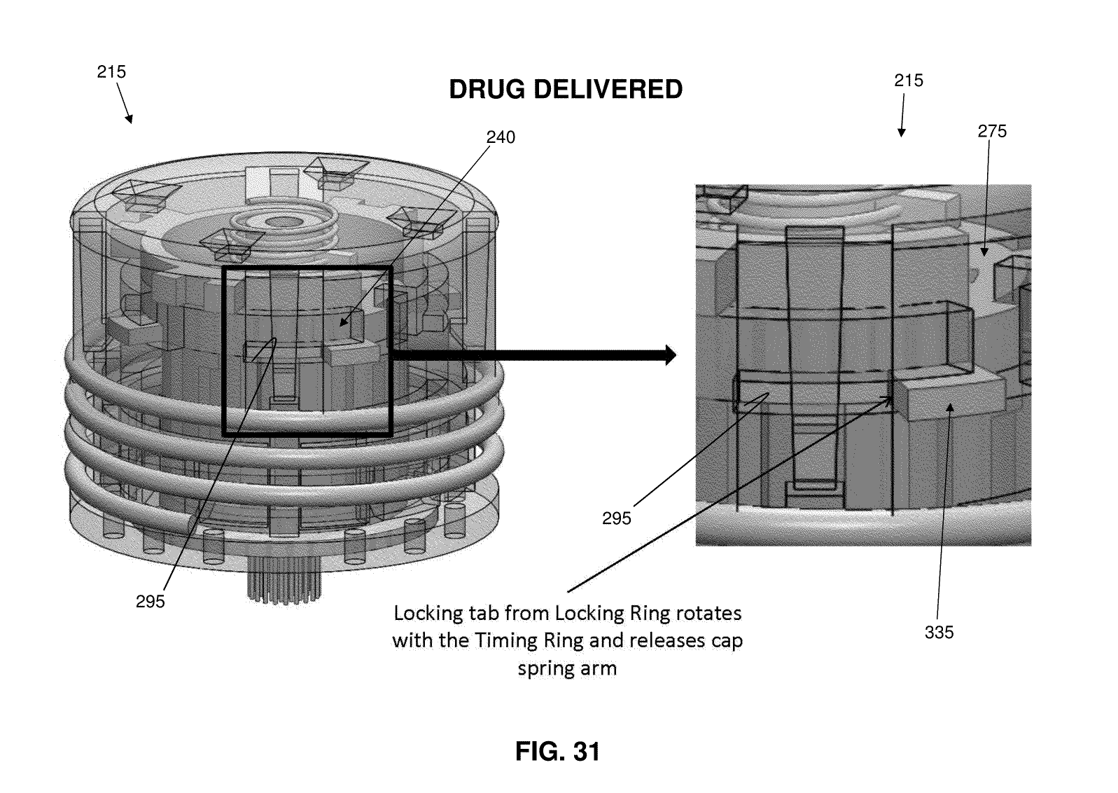

Looking next at FIGS. 15-33, there is shown another preferred form of the present invention. This form of the present invention utilizes the rigid, non-porous hollow nano-needle 105 of FIGS. 12A-12E, 13 and 14, combined with a modified form of deployment apparatus.

More particularly, and looking now at FIGS. 15-19, there is provided a nano-syringe system 205 comprising a carrier 210 having a syringe 215 carried thereby, wherein syringe 215 comprises a plurality of nano-needles 105 comprising a plurality of hollow fibers (e.g., CNTs) 110. Carrier 210 comprises a flexible concave member 220 having syringe 215 mounted within its concavity 225. The remainder of the volume of concavity 225 is filled with a gel 230. Holes 231 formed in carrier 210 allow visualization of the top of syringe 215 as will hereinafter be discussed. Preferably, a peel-away strip 235 covers the bottom surface of flexible concave member 220, sealing syringe 215 and gel 230 in concavity 225 of flexible concave member 220 until the time of use.

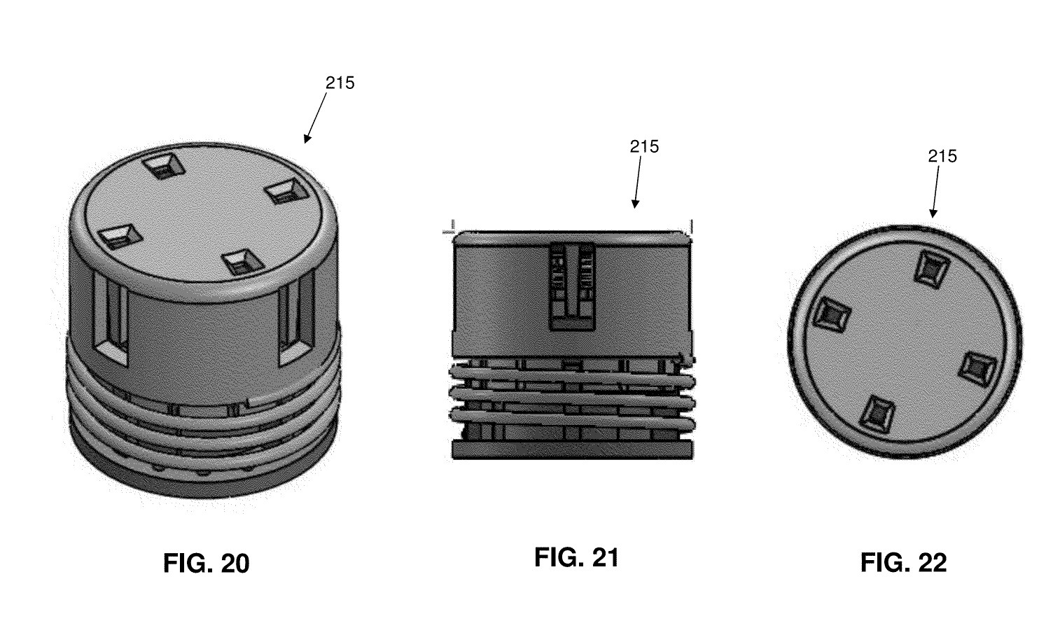

Looking next at FIGS. 20-24, syringe 215 generally comprises a hollow base 240; a nano-needle assembly 245 movably mounted within hollow base 240; a plunger rod 250 and a plunger disc 255 adapted for distal movement within hollow base 240; a cap 260 secured to plunger rod 250 for selectively advancing plunger rod 250 and plunger disc 255 distally within hollow base 240; a cap spring 265 for biasing cap 260 proximally; a locking ring 270, a timing ring 275, and a plunger spring 280 for controlling the cycling of plunger rod 250 and plunger disc 255 within hollow base 240; and a cycle indicator 285 for indicating the cycle status of syringe 215, all as will hereinafter be discussed.

More particularly, hollow base 240 comprises L-shaped slots 290, a lip 295 and fingers 300. Nano-needle assembly 245 comprises a plurality of nano-needles 105 mounted to a wafer substrate 305 having a plurality of openings 307, and a needle support plate 310 secured to the distal end of hollow base 240 and including a plurality of openings 315 for permitting nano-needles 105 to extend therethrough. Cap 260 comprises tabs 320 for locking onto lip 295 of hollow base 240, tabs 323 for selectively engaging locking ring 270, and windows 325 for allowing visualization of cycle indicator 285, whereby to identify the cycle status of syringe 215.

Locking ring 270 comprises slots 330 for engagement with cycle indicator 285, tabs 335 for engagement in L-shaped slots 290 of base 240 and for selective engagement by tabs 323 of cap 260, and fingers 340 for engaging timing ring 275. Timing ring 275 comprises longitudinally-extending slots 345 for receiving fingers 340 of timing ring 275, and keyways 350 for receiving fingers 300 of base 240. As a result of this construction, locking ring 270 and timing ring 275 are rotationally fixed to one another, but are able to telescope relative to one another; and timing ring 275 is able to move both rotationally and telescopically relative to base 240, but only as permitted by the engagement of fingers 300 in keyways 350. Plunger spring 280 is a torsion compression spring, biasing timing ring 275 both distally and rotationally, as will hereinafter be discussed. It will also be appreciated that plunger disc 255 is mounted to timing ring 275 such that plunger disc 255 moves with timing ring 275 moves, as will hereinafter be discussed.

Cycle indicator 285 comprises legs 355 for seating in slots 330 of locking ring 270, whereby to couple rotation of cycle indicator 285 with rotation of locking ring 270, and includes color coding 360, 365 on its upper surface for visualization through windows 325 of cap 260 (and through holes 231 in carrier 210).

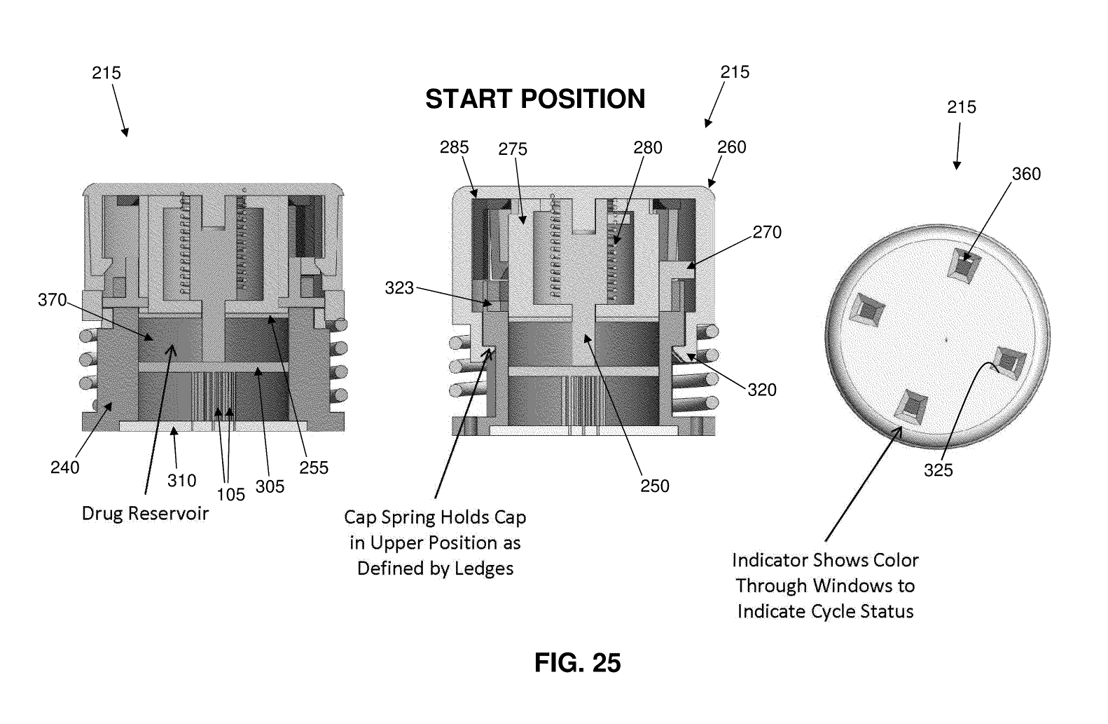

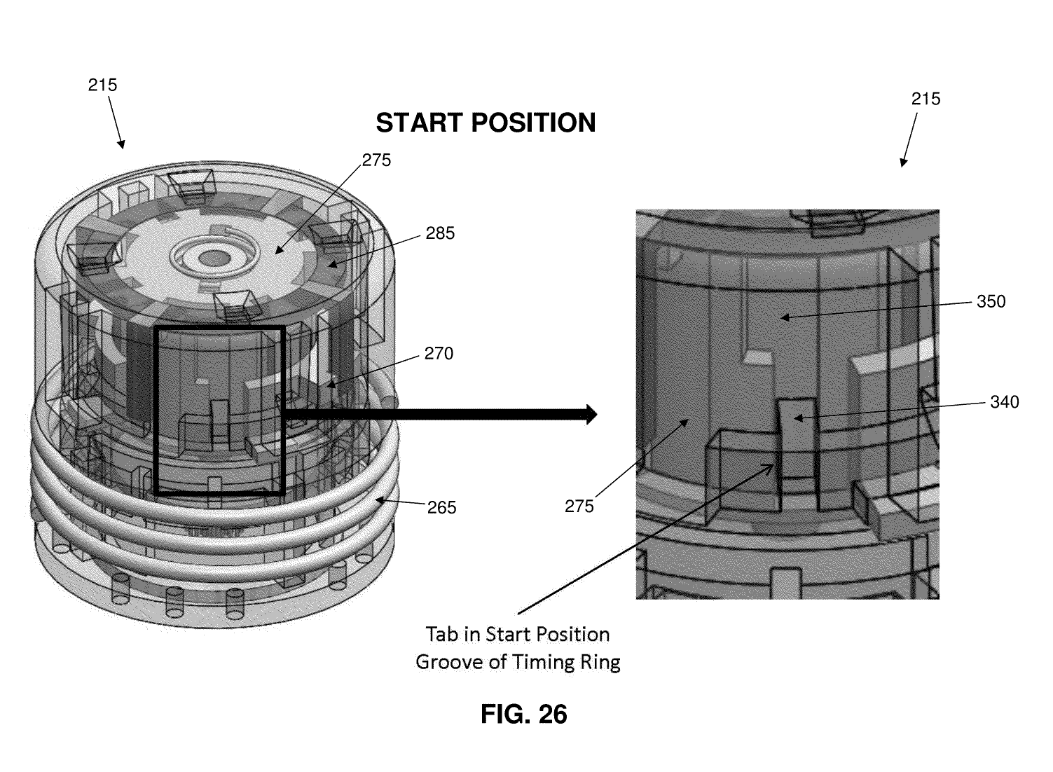

With this form of the invention, prior to use, and looking now at FIGS. 25 and 26, nano-needle assembly 245 is disposed within hollow base 240 so that its wafer substrate 305 is disposed intermediate needle support plate 310 and plunger disc 255, with the distal tips of nano-needles 105 extending into openings 315 in needle support plate 310. The active agent to be injected into the patient resides in the chamber 370 defined between wafer substrate 305 and plunger disc 255.

When nano-syringe system 205 is to be used to inject the active agent into a patient, peel-away strip 235 is removed from the bottom surface of flexible concave member 220, and the bottom of the system is placed against the skin of the patient at the active agent delivery site.

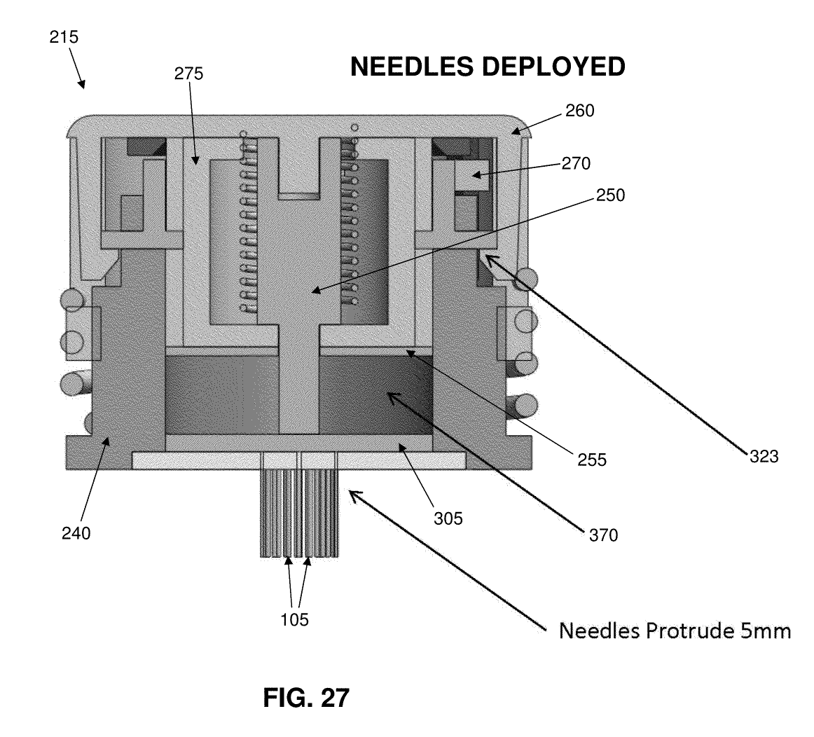

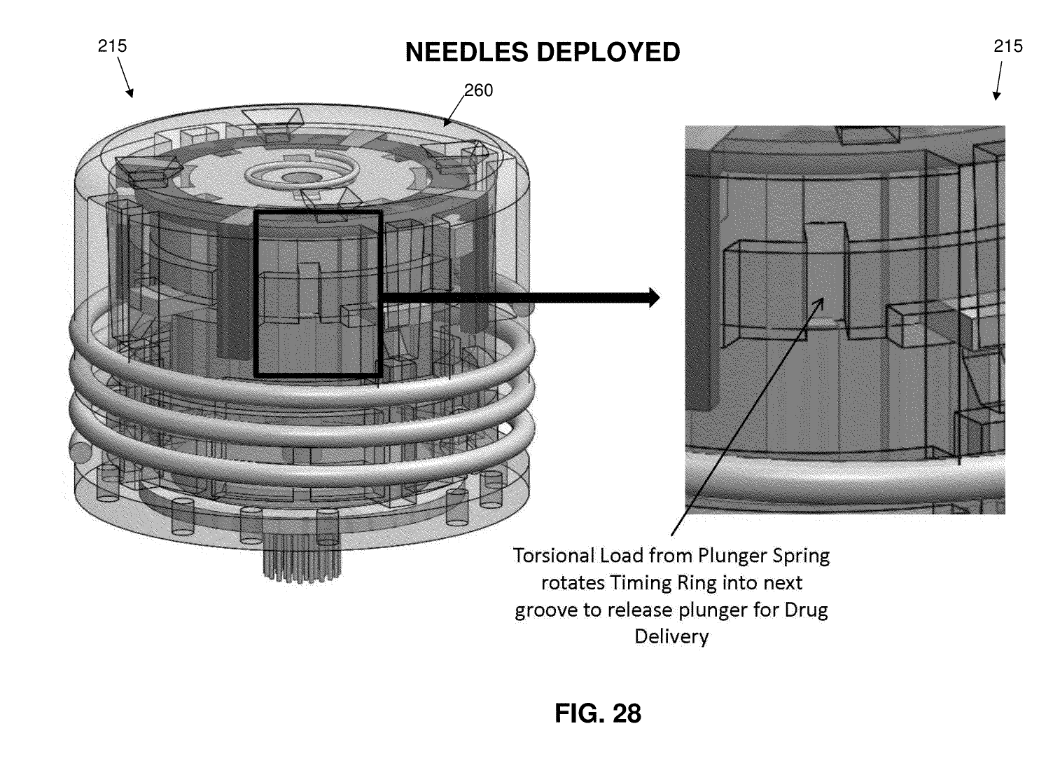

Next, and looking now at FIGS. 27 and 28, cap 260 is depressed. This action causes plunger rod 250, plunger disc 255 and wafer substrate 305 to move distally as a unit, carrying chamber 370 distally within hollow base 240 while preserving its volume. As wafer substrate 305 moves distally within base 240, nano-needles 105 advance through openings 315 in needle support plate 310 and enter the skin of the patient. Distal movement of nano-needles 105 continues until wafer substrate 305 seats against needle support plate 310. Note that at this point in the operation of nano-syringe system 205, plunger disc 255 has not advanced with respect to wafer substrate 305, and hence none of the active agent in chamber 370 has been ejected from nano-needles 105.

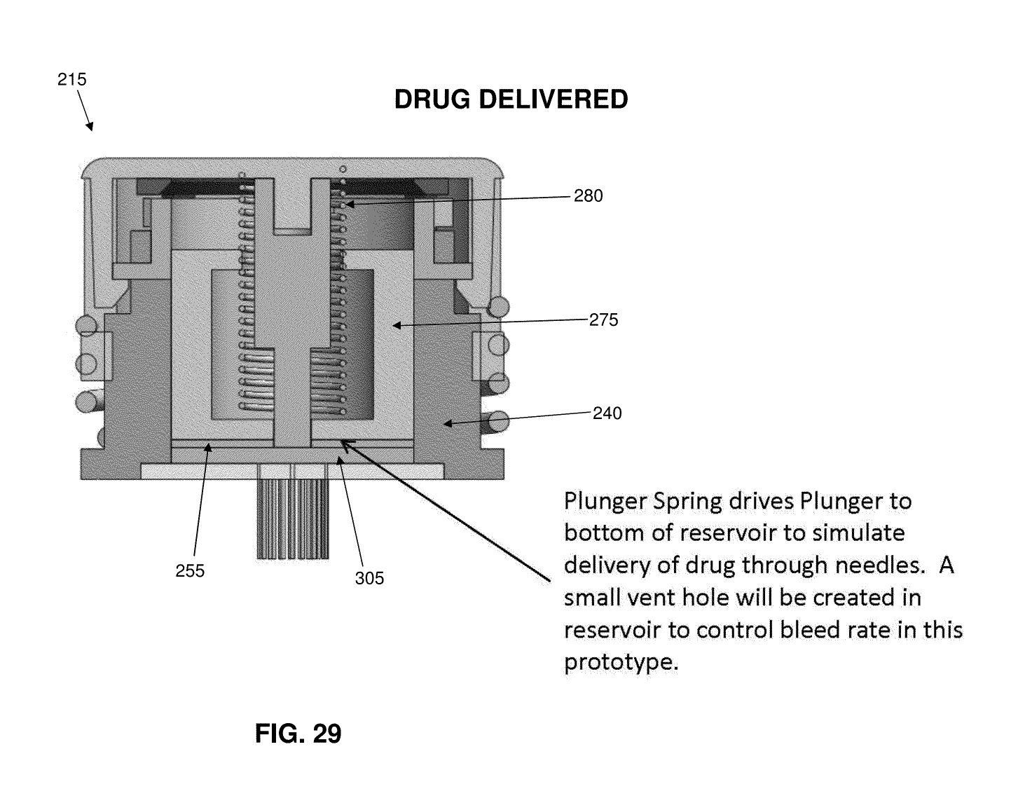

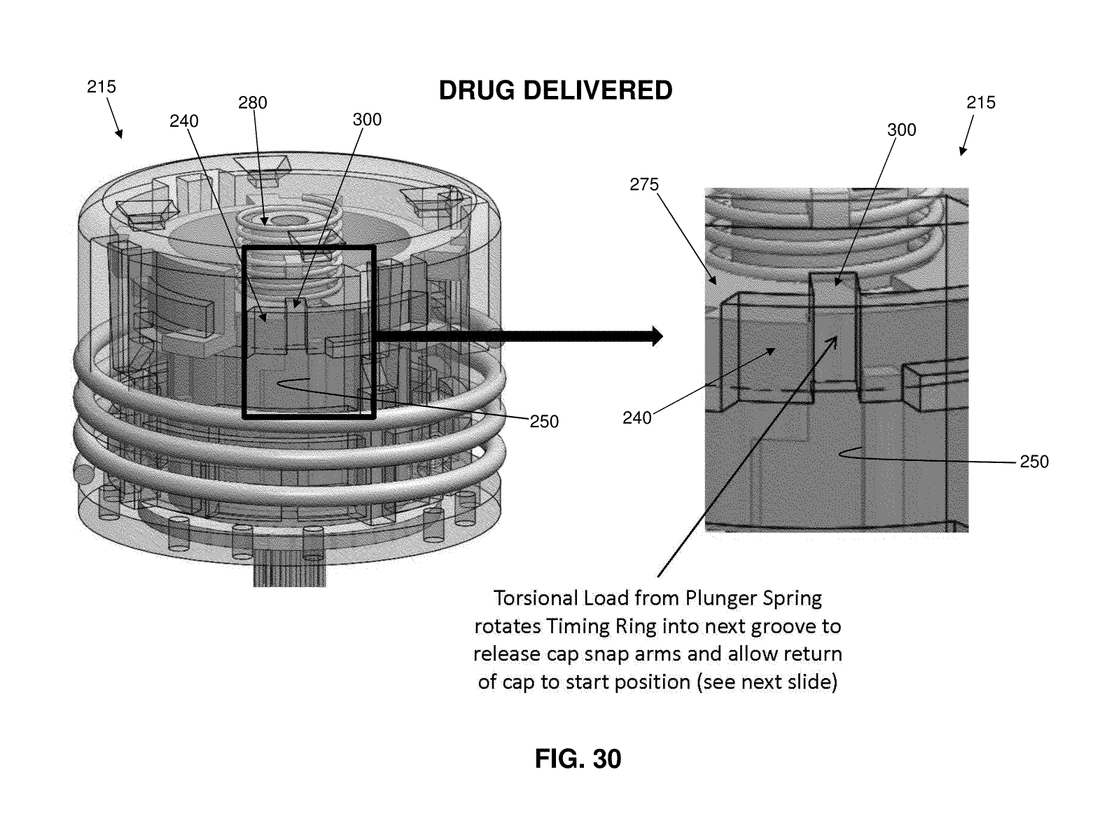

At the same time, the downward movement of cap 260 causes plunger rod 250 to move timing ring 275 distally. When this occurs, plunger spring 280, which is both a torsion and compression spring, causes timing ring 275 to rotate, which causes locking ring 270 to also rotate (by virtue of the engagement of fingers 340 in longitudinally-extending slots 345 of timing ring 275). As a result, plunger spring 280 also moves timing ring 275 distally within hollow base 240, causing plunger disc 255 to move distally until it engages wafer substrate 305. Distal movement of plunger disc 255 forces the active agent residing in chamber 370 distally, through openings 307, into nano-needles 105 and into the patient. See FIGS. 29 and 30.

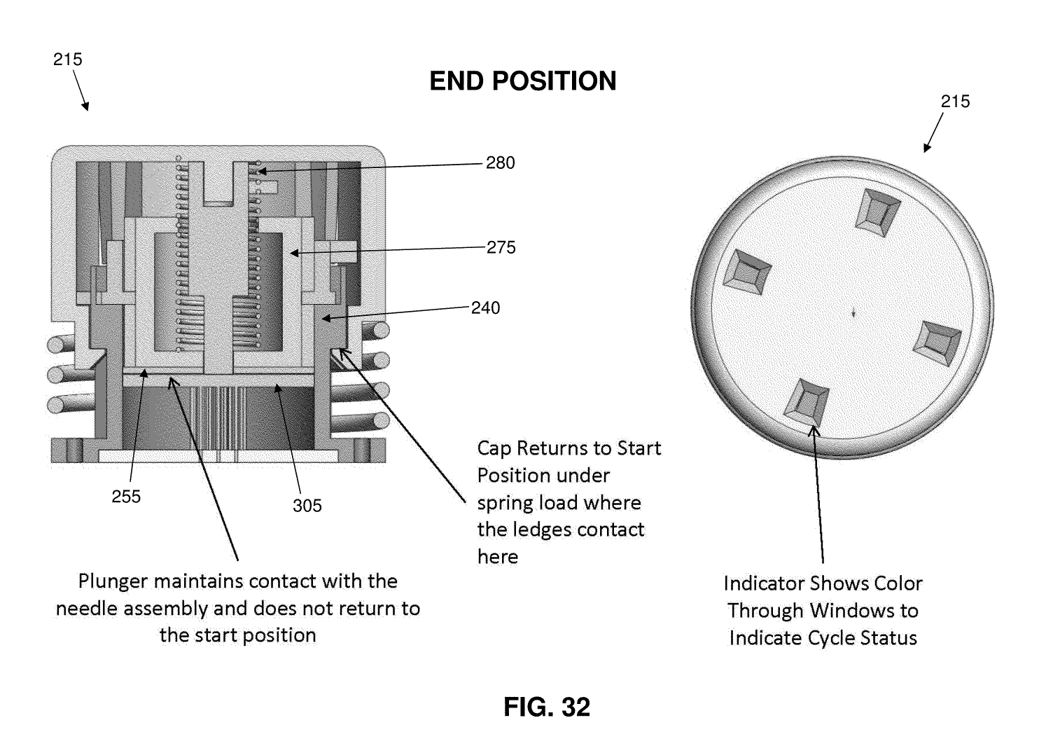

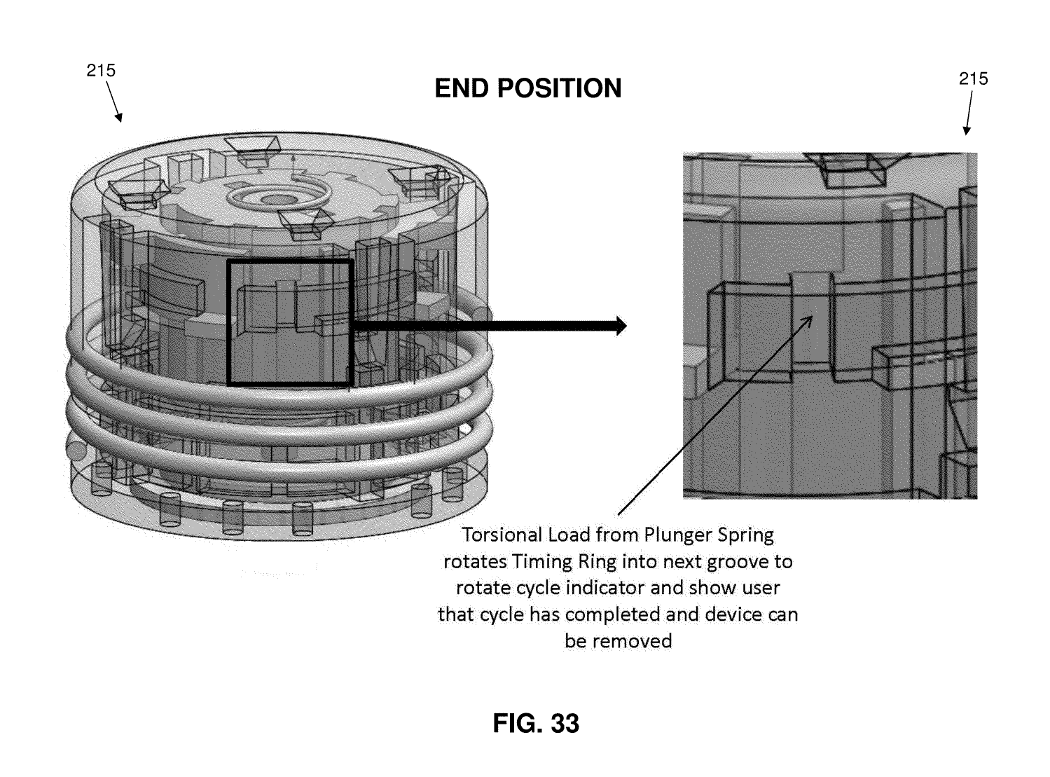

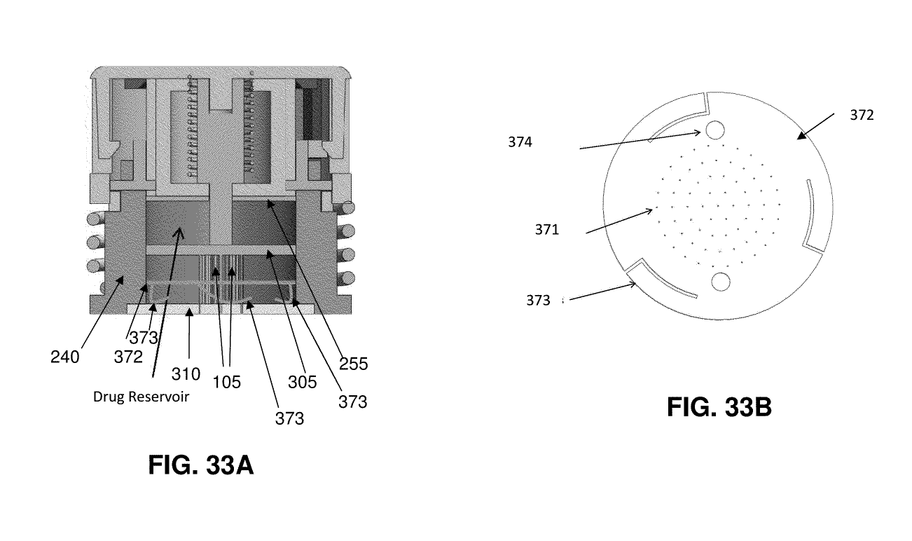

When plunger spring 280 has moved timing ring 275 distally a sufficient distance to cause plunger disc 255 to move distally and engage wafer substrate 305 (and hence eject the active agent into the patient), the torsional force of plunger spring 280 causes timing ring 275 to rotate, whereby to rotate locking ring 270. Rotation of locking ring 270 causes tabs 335 to move within L-shaped slot 290, whereby to release tabs 323 of cap 260. When tabs 323 are released from engagement with tabs 335, cap 260 is moved proximally by cap spring 265. Proximal movement of cap 260 causes proximal movement of plunger rod 250 and hence proximal movement of wafer substrate 305 and nano-needles 105, whereby to withdraw nano-needles 105 from the skin of the patient. See FIGS. 32 and 33.

With this form of the invention, and looking now at FIGS. 33A and 33B, it can be desirable to provide a spring-biased needle guide plate 372 between wafer substrate 305 and needle support plate 310, so as to prevent buckling of the nano-needles 105. More particularly, in this form of the invention, spring-biased needle guide plate 372 comprises spring legs 373 which serve to spring-support spring-biased needle guide plate 372 above needle support plate 310. In one form of the invention, legs 373 are formed out of a portion of spring-biased needle guide plate 372 and bent out of the plane of spring-biased needle guide plate 372 so as to provide spring support for spring-biased needle guide plate 372 above needle support plate 310. Spring-biased needle guide plate 372 comprises openings 371 for permitting nano-needles 105 to extend from wafer substrate 305, through spring-biased needle guide plate 372, and through openings 315 in needle support plate 310. Additional holes 374 enable alignment of the guide plate 372 during assembly. Thus it will be seen that with this form of the invention, nano-needle assembly 245 is disposed within base 240 so that its wafer substrate 305 is disposed intermediate needle support plate 310 and plunger disc 255, with the distal tips of nano-needles 105 extending through openings 371 in spring-biased needle guide plate 372 and then extending into openings 315 in needle support plate 310. The spring-biased needle guide plate 372 serves as a moving support plate to prevent buckling of the nano-needles 105 during advancement of nano-needles 105, i.e., as wafer substrate 305 moves towards needle support plate 310, wafer substrate 305 engages spring-biased needle guide plate 372 and forces it distally, against the power of spring legs 373, until spring-biased needle guide plate 372 effectively engages needle support plate 310. During this “power stroke”, spring-biased needle guide plate 372 serves as a moving support plate moving along nano-needles 105 to prevent buckling of the nano-needles 105 during advancement of nano-needles 105.

### Alternative Needle Constructions

In the foregoing description, nano-syringe system 5 comprises a carrier 10 having a syringe 15 carried thereon, wherein syringe 15 comprises a plurality of hollow fibers 20.

Carbon nanotubes (CNTs) were used as an exemplary material for the construction of the nano-needles, but, if desired, hollow CNTs 20 may be replaced by alternative tubular structures.

Furthermore, in the foregoing description, nano-needle 105 comprises a plurality of CNTs 110 extending out of wafer substrate 115 and arranged so as to collectively form a hollow tubular meta-structure 120 having a lumen 125 defined thereby, with hollow tubular meta-structure 125 thereafter being sealed so as to form nano-needle 105.

However, if desired, CNTs 110 may be replaced by alternative tubular structures.

Also, in the foregoing description, nano-syringe system 205 comprises a carrier 210 having a syringe 215 carried thereon, wherein syringe 215 comprises a nano-needle 105 comprising a plurality of hollow CNTs 110.

Again, if desired, CNTs 110 may be replaced by alternative tubular structures.

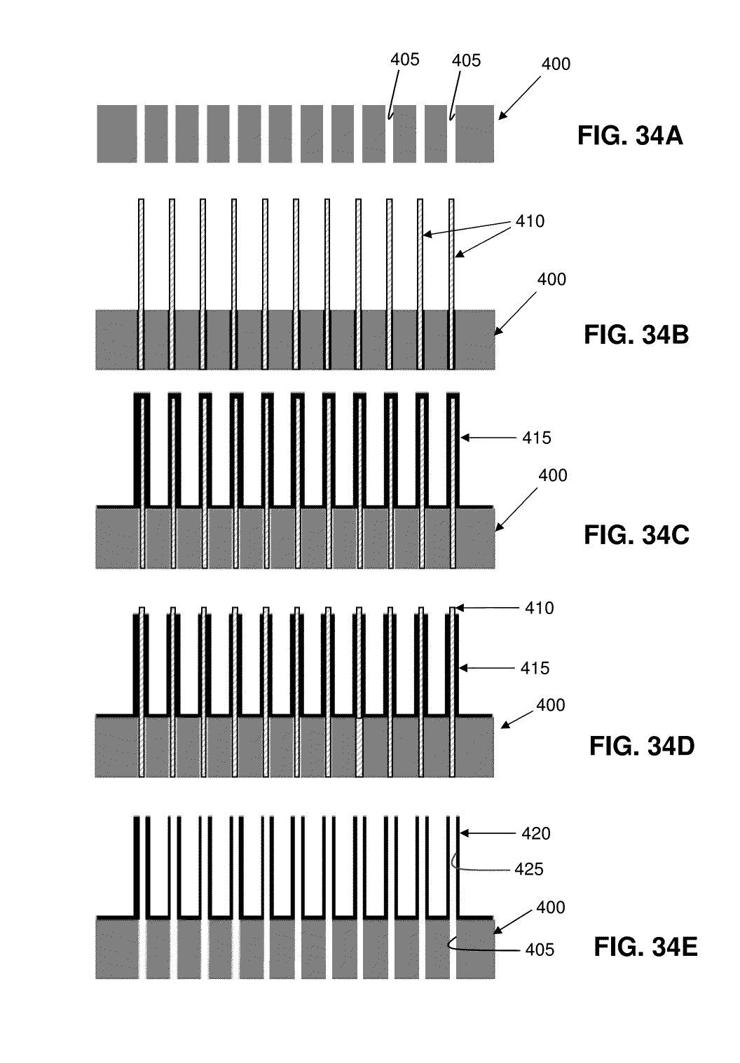

By way of example but not limitation, CNTs 20 and/or CNTs 110 may be replaced by tubular structures formed using the process shown in FIGS. 34A-34E. More particularly, with this process, a support plate 400, having holes 405 extending therethrough, is provided (FIG. 34A). Fibers 410 are inserted into, and fixed to, support plate 400 such that each fiber is supported and freestanding, with spacing between adjacent fibers (FIG. 34B). Fibers 410 are then overcoated with a stiff material 415 (FIG. 34C). This fiber overcoating process may utilize any one of several common coating processes, including chemical vapor deposition, plating, physical vapor deposition (sputtering or evaporation), atomic layer deposition, spraying, dipping, electrophoretic deposition or the like. Fixation may include sintering, heat treating, solvent welding, etc. The stiff material 415 overcoating the free ends of fibers 410 is then removed, whereby to expose fibers 410 (FIG. 34D). Fibers 410 are then selectively etched away, without etching stiff material 415, whereby to leave hollow tubes 420 of stiff material 415 extending out of support plate 400, with the lumens 425 of hollow tubes 420 communicating with holes 405 in support plate 400 (FIG. 34E).

Various materials consistent with this approach may be used to form support plate 400, fibers 410, stiff material 415 and the preferential etchant. Of course, the selections of these materials must be coordinated with one another so as to be consistent with this fabrication process.

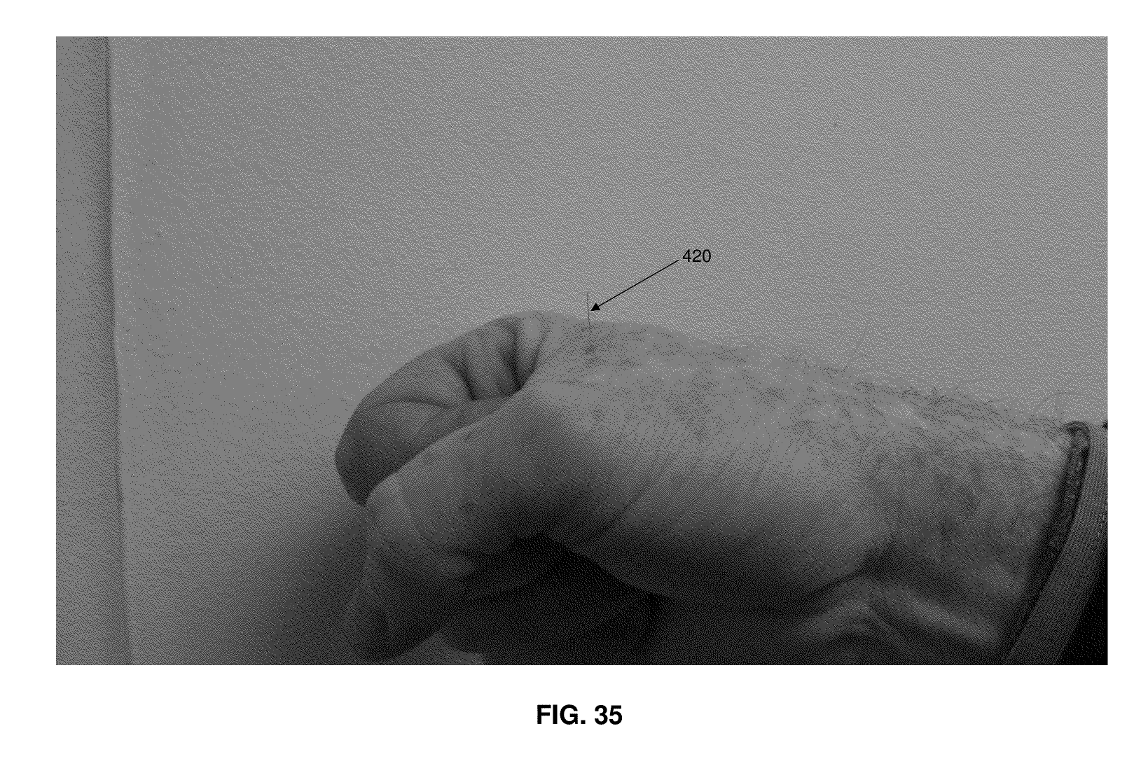

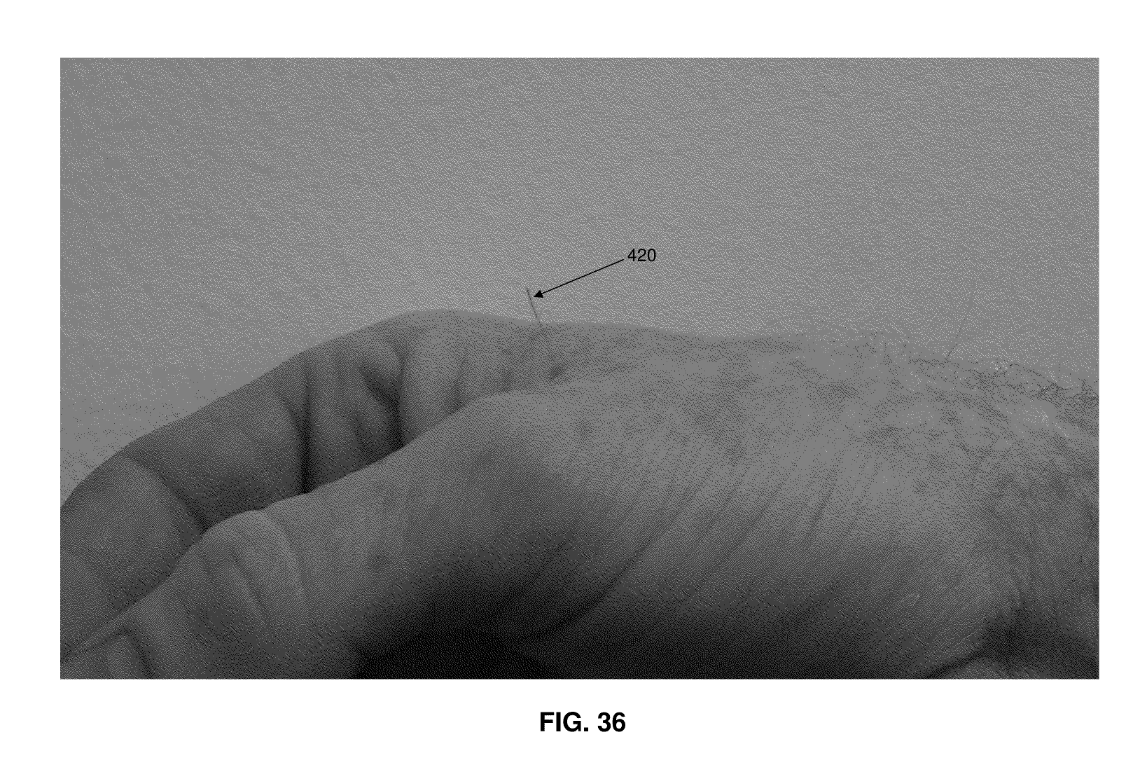

By way of example but not limitation, in one preferred form of the invention, stiff material 415 comprises tungsten, whereby to form tungsten hollow tubes 420. In this form of the invention, support plate 400 may comprise an etch-resistant material, fibers 410 may comprise plastics, glass, a ceramic, a low melting metal, or a readily etchable metal, and the preferential etchant may comprise hydrofluoric acid for the glass fibers, or a solvent for the plastic fibers. FIGS. 35 and 36 show an exemplary tungsten hollow tube 420, formed in accordance with the process depicted in FIGS. 34A-34E, extending out of the skin of a patient.

By way of further example but not limitation, in another preferred form of the invention, stiff material 415 comprises alumina, whereby to form alumina hollow tubes 420. In this form of the invention, support plate 400 may comprise either a plastic or a ceramic, fibers 410 may comprise plastic, glass or metals, and the preferential etchant may comprise solvents for plastic fibers, or HF for glass fibers, or HCl for ferrous metal fibers.

In general, it is preferred that support plate 400 comprises one from the group consisting of stainless steel or another metal, plastics or ceramics.

In general, it is preferred that fibers 410 comprise at least one from the group consisting of glass, carbon or a ceramic.

In general, it is preferred that stiff material 415 comprises at least one from the group consisting of a metal, ceramic or diamond-like carbon.

In general, it is preferred that the preferential etchant comprises at least one from the group consisting of 1:1 HF:HNO₃; 1:1 HF:HNO₃ (thin films); 3:7 HF:HNO₃; 4:1 HF:HNO₃ (rapid attack); 1:2 NH₄OH:H₂O₂ (thin films good for etching tungsten from stainless steel, glass, copper and ceramics, will also etch titanium as well); 305 g:44.5 g:1000 ml K₃Fe(CN)₆:NaOH:H₂O (rapid etch); HCl (slow etch, dilute or concentrated); HNO₃ (very slow etch, dilute or concentrated); H₂SO₄ (slow etch, dilute or concentrated); HF (slow etch, dilute or concentrated); H₂O₂; 1:1, 30%:70%, or 4:1 HF:HNO₃; 1:2 NH₄OH:H₂O₂; 4:4:3 HF:HNO₃:HAc; CBrF₃ RIE etch; 305 g:44.5 g:1000 ml K₃Fe(CN)₆:NaOH:H₂O (very rapid etch); HCl solutions (slow attack); HNO₃ (slight attack) Aqua Regia 3:1 HCL:HNO₃ (slow attack when hot or warm); H₂SO₄ dilute and concentrated (slow etch); HF dilute and concentrated (slow etch); and Alkali with oxidizers (KNO₃ and PbO₂) (rapid etch).

### Example 1

A roving of 15 micron diameter glass filament was debundled into individual filaments and processed in a chemical vapor deposition chamber. A tungsten coating, 20 microns thick, was deposited on the filaments, leading to the growth in the diameter of the filaments to 55 microns. The coated filaments were then cut to length, and immersed in an HF bath for several days. The disparity in the etch rates of tungsten and glass by hydrofluoric acid enables the glass core to be etched out, leaving the tungsten intact. However, the process is retarded by the limited area of glass exposed to the acid. Once etched, one end of each tungsten hollow needle was placed into holes in a Lexan support plate, so that each hollow needle was vertically oriented and freestanding. The solvent dicholoromethane was used to solvent-weld the tungsten tubes to the Lexan.

### Example 2

As the individual handing required in Example 1 was arduous, a second process was developed to process the filaments in parallel. A length of 15 micron OD glass fiber roving was debundled and one end of each fiber was inserted into a stainless steel support plate, 0.1 mm thick, which had been laser drilled with 15 micron holes to receive the fibers. The plate thickness to hole diameter ratio in this case is approximately 6.6:1, which has been found sufficient to fixate the filaments, and within the capability of laser drilling. The glass fibers were then overcoated with tungsten by a CVD process, which also covered the stainless support plate, all to a thickness of 20 microns. The backside was protected to prevent coating on the backside of the support plate. The tungsten coating at the fiber tips was exposed to an etchant, (K₃Fe(CN)₆:NaOH:H₂O 30.5 g:4.45 g:100 ml) to re-expose the glass fibers. The glass fibers were then etched out with hydrofluoric acid, leaving an array of hollow needles, vertically standing where their glass fiber cores had once been. The process followed in this example is illustrated in FIGS. 34A-34E.

### Example 3

Lengths of 15 micron palladium wire were passed through a copper coated polyimide support sheet, such that each wire protruded from the support plate by 5 mm on the metallized side, and protruded by a smaller amount on the side without the metallization. The palladium wires and copper surface were dipped into an alumina ceramic slurry and a DC voltage was applied to cause electrophoretic deposition on the copper and wires, which served as the cathode. The polyimide support was then removed, leaving a ceramic deposit both where the metallized polyimide had been, and also around the wires. The wires were carefully removed, and the ceramic article sintered to create a plate with hollow needles. The needles were not universally open after this process, so the article was potted in a wax, then polished on a silicon carbide paper to expose the inner diameter. The wax was then removed, leaving the article with the holes exposed.

### Active Agents

The system delivers therapeutic active agents with pharmacologic and pharmacokinetic profiles similar to, or better than, dosing with original modes of delivery, e.g., via intramuscular injection (IM), subcutaneous injection (SC), intravenous administration (IV), suppository administration, oral and/or nasal delivery, etc., often with reduced toxicities (which may correlate to the reduced levels of therapeutic active agents or decreased need for hepatic metabolism, or a combination thereof).

The pharmacological active agents can include any molecule or composition in any number or amount. Preferably, the active agent may be used individually or in combination with one another.

Classes of active agents can include analgesics (acetaminophen, cox-1 and/or cox-2 inhibitors, ibuprofen, lidocaine, etc.); emergency medications (e.g., epinephrine, atropine, 17-(cyclopropylmethyl)-4,5α-epoxy-3,14-25 dihydroxymorphinan-6-one (naltrexone and flumazenil)); anti-arrhythmics (e.g., amiodarone, diltiazem and atropine); lipid modulators (e.g., statins, atorvastatin, etc.); anti-convulsants (e.g., phenytoin sodium, topiramate, oxcarbazepine, etc.); anti-coagulants (e.g., low molecular weight heparin, enoxaparin, etc.); vitamins and nutritional supplements (e.g., co-enzyme Q10, etc.); corticosteroids (e.g., prednisone); oncology agents (e.g., paclitaxel, carboplatin, etc.); centrally active agents (e.g., sumatriptan); micropeptides (e.g., Xen 2174); rheumatologic agents (e.g., etanercept, adalimumab, etc.); bone marrow stimulators (e.g., filgrastim, erythropoetin alfa, etc.); osteoporosis medications (e.g., teriparatide); growth factors (e.g., somatotropin); immune modulators (e.g., cyclophosphamide, tacrolimus, mycophenylate mofetil, azathioprine, etc.); anti-human antibodies (e.g., gamma globulin); central and peripheral neuromuscular disorder agents (e.g., glatirameracetate); endocrines (e.g., human growth hormone (HGH), profasi (HCG analogue), insulin and/or insulin analogues, etc.), and vascular tone modulators (e.g., sildenafil). An additional example of an active agent may include PT-141.

Exemplary active agents include agents for treating infections such as antibacterial, anti-fungal and antibiotic agents; agents for treating cardiovascular conditions such as chlorothiazide (diuretic), propranolol (antihypertensive), hydralazine (peripheral vasodilator), isosorbide or nitroglycerin (coronary vasodilators), metoprolol (beta-blocker), procainamide (antiarrythmic), clofibrate (cholesterol reducer) or coumadin (anticoagulant); agents for treating internal conditions such as conjugated estrogen (hormone), tolbutamide (antidiabetic), levothyroxine (thyroid conditions), propantheline (antispasmodic), cimetidine (antacid), phenyl propanolamine (anti-obesity), atropine or diphenoxalate (antidiarrheal agents), docusate (laxative), or prochlorperazine (antinauseant); agents for treating mental health conditions such as haloperidol or chlorpromazine (tranquilizers), doxepin (psychostimulant), phenytoin (anticonvulsant), levo dopa (anti-parkinism), benzodiazepine (anti-anxiety) or phenobarbital (sedative); agents for treating inflammation (anti-inflammatories) such as fluorometholone, acetaminophen, phenacetin, aspirin, hydrocortisone, or predisone; agents for treating allergic reactions (antihistamines) such as diphenhydramine hydrochloride or dexchlorpheniramine maleate; agents for treating infection (antibiotics) such as sulfanilamide, sulfamethizole, tetracycline hydrochloride, penicillin and its derivatives, cephalosporin derivatives or erythromycin; agents for providing chemotherapy (chemotherapeutic agents) such as sulfathiazole, doxorubicin, cisplatin or nitrofurazone; agents for providing local pain relief (topical anaesthetics) such as benzocaine; agents for treating cardiovascular disorders (cardiac tonics) such as digitalis or digoxin; agents for treating pulmonary distress (antitussives and expectorants) such as codeine phosphate, dextromethorphan or isoproterenol hydrochloride; agents for treating oral conditions (oral antiseptics) such as chlor hexidine hydrochloride or hexylresorcinol; agents for providing enzymes such as lysozyme hydrochloride or dextronase; birth control agents such as estrogen; agents for treating ophthalmic disorders such as timolol or gentamycin, and the like. In addition, active agents may also include whole proteins such as the VP3 capsid protein (also known as the VPThr and VP1 capsid proteins in other nomenclature systems), insulin or interferon; polypeptide treating agents such as endorphins, human growth hormone or bovine growth hormone, or still lower molecular weight polypeptides or conjugates of those polypeptides, linked protein carriers, etc.

The system can, optionally deliver an effective amount of active agents selected from at least one of an anti-infective, a cardiovascular system drug, a central nervous system drug, an autonomic nervous system drug, a respiratory tract drug, a gastrointestinal (GI) tract drug, a hormonal drug, a drug for fluid or electrolyte balance, a hematologic drug, an antineoplactic drug, an immunomodulation drug, an ophthalmic drug, an otic or nasal drug, a topical drug, a nutritional drug, a statin, or the like. Active agents can also be at least one selected from nonnarcotic analgesics or at least one selected from the group consisting of antipyretics, nonsteroidal anti-inflammatory drugs, narcotics or at least one opioid analgesics, sedative-hypnotics, anticonvulsants, antidepressants, antianxiety drugs, antipsychotics, central nervous system stimulants, antiparkinsonians, and miscellaneous central nervous system drugs. The active agent can be at least one selected from the group consisting of cholinergics (parasympathomimetics), anticholinergics, adrenergics (sympathomimetics), adrenergic blockers (sympatholytics), skeletal muscle relaxants, and neuromuscular blockers. Nonnarcotic analgesics or antipyretics can be at least one selected from the group consisting of acetaminophen, asprin, choline magnesium trisalicylate, diflunisal, and magnesium salicylate.

Nonsteroidal anti-inflammatory drugs can be at least one selected from the group consisting of celecoxib, diclofenac potassium, diclofenac sodium, etodolac, fenoprofen calcium, flurbiprofen, ibuprofen, indomethacin, ketoprofen, ketorolac tromethamine, nabumetone, naproxen, naproxen sodium, oxaprozin, piroxicam, rofecoxib, and sulindac. Narcotic or opioid analgesics can be at least one selected from the group consisting of alfentanil hydrochloride, buprenorphine hydrochloride, butorphanol tartrate, codeine phosphate, codeine sulfate, fentanyl citrate, fentanyl transdermal system, fentanyl transmucosal, hydromorphone hydrochloride, meperidine hydrochloride, methadone hydrochloride, morphine hydrochloride, morphine sulfate, morphine tartrate, nalbophine hydrochloride, oxycodone hydrochloride, oxycodone pectinate, oxymorphone hydrochloride, pentazocine hydrochloride, pentazocine hydrochloride and naloxone hydrochloride, pentazocine lactate, propoxyphene hydrochloride, propoxyphene napsylate, remifentanil hydrochloride, sufentanil citrate, and 25 tramadol hydrochloride.

A sedative-hypnotic agent can be at least one selected from the group consisting of chloral hydrate, estazolam, flurazepam hydrochloride, pentobarbital, pentobarbital sodium, phenobarbital sodium, secobarbital sodium, temazopam, triazolam, zaleplon, and zolpidem tartrate.

An anticonvulsant agent can be at least one selected from the group consisting of acetazolamide sodium, carbamazepine, clonazepam, clorazepate dipotassium, diazepam, divalproex sodium, ethosuximde, fosphenytoin sodium, gabapentin, lamotrigine, magnesium sulfate, phenobarbital, phenobarbital sodium, phenytoin, phenytoin sodium, phenytoin sodium (extended), primidone, tiagabine hydrochloride, topiramate, valproate sodium, and valproic acid.

An antidepressant agent can be at least one selected from the group consisting of amitriptyline hydrochloride, amitriptyline pamoate, amoxapine, bupropion hydrochloride, citalopram hydrobromide, clomipramine hydrochloride, desipramine hydrochloride, doxepin hydrochloride, fluoxetine hydrochloride, imipramine hydrochloride, imipramine pamoate, mirtazapine, nefazodone hydrochloride, nortriptyline hydrochloride, paroxetine hydrochloride, phenelzine sulfate, sertraline hydrochloride, tranylcypromine sulfate, trimipramine maleate, and venlafaxine hydrochloride.

An antianxiety agent can be at least one selected from the group consisting of alprazolam, buspirone hydrochloride, chlordiazepoxide, chlordiazepoxide hydrochloride, clorazepate dipotassium, diazepam, doxopin hydrochloride, hydroxyzine embonate, hydroxyzine hydrochloride, hydroxyzine pamoate, lorazepam, mephrobamate, midazolam hydrochloride, and oxazopam.

An antipsychotic agent can be at least one selected from the group consisting of chlorpromazine hydrochloride, clozapine, fluphenazine decanoate, fluephenazine enanthate, fluphenazine hydrochloride, haloperidol, haloperidol decanoate, haloperidol lactate, loxapine hydrochloride, loxapine succinate, mesoridazine besylate, molindone hydrochloride, olanzapine, perphenazine, pimozide, prochlorperazine, quetiapine fumarate, risperidone, thioridazine hydrochloride, thiothixene, thiothixene hydrochloride, and trifluoperazine hydrochloride.

A central nervous system stimulant agent can be at least one selected from the group consisting of amphetamine sulfate, caffeine, dextroamphetamine sulfate, doxapram hydrochloride, methamphetamine hydrochloride, methylphenidate hydrochloride, modafinil, pemoline, and phentermine hydrochloride.

An anti-parkinsonian agent can be at least one selected from the group consisting of amantadine hydrochloride, benztropine mesylate, biperiden hydrochloride, biperiden lactate, bromocriptine mesylate, carbidopa-levodopa, entacapone, levodopa, pergolide mesylate, pramipexole dibydrochloride, ropinirole hydrochloride, selegiline hydrochloride, tolcapone, and trihexyphenidyl hydrochloride.

A central nervous system active agent can be at least one selected from the group consisting riluzole, bupropion hydrochloride, donepezil hydrochloride, droperidol, fluvoxamine maleate, lithium carbonate, lithium citrate, naratriptan hydrochloride, nicotine polacrilex, nicotine transdermal system, propofol, rizatriptan benzoate, sibutramine hydrochloride monohydrate, sumatriptan succinate, tacrine hydrochloride, duoxetene, milnaciprin, gabapentin, pregabalin and zolmitriptan.

A cholinergic (e.g., parasymathomimetic) active agent can be at least one selected from the group consisting of bethanechol chloride, edrophonium chloride, neostigmine bromide, neostigmine, methylsulfate, physostigmine salicylate, and pyridostigmine bromide.

A anticholinergics agent can be at least one selected from the group consisting of atropine sulfate, dicyclomine hydrochloride, glycopyrrolate, hyoscyamine, hyoscyamine sulfate, propantheline bromide, scopolamine, scopolamine butylbromide, and scopolamine hydrobromide.

An adrenergic (sympathomimetics) active agent can be at least one selected from the group consisting of dobutamine hydrochloride, dopamine hydrochloride, metaraminol bitartrate, norepinephrine bitartrate, phenylephrine hydrochloride, pseudoephedrine hydrochloride, and pseudoephedrine sulfate.

An adrenergic blocker 1 (sympatholytic) agent can be at least one selected from the group consisting of dibydroergotamine mesylate, ergotamine tartrate, methysergide maleate, and propranolol hydrochloride.

A skeletal muscle relaxant agent can be at least one selected from the group consisting of baclofen, carisoprodol, chlorzoxazone, cyclobenzaprine hydrochloride, dantrolene sodium, methocarbamol, and tizanidine hydrochloride.

A neuromuscular blocker active agents can be at least one selected from the group consisting of atracurium besylate, cisatracurium besylate, doxacurium chloride, mivacurium chloride, pancuronium bromide, pipecuronium bromide, rapacuronium bromide, rocuronium bromide, succinylcholine chloride, tubocurarine chloride, and vecuronium bromide.

An anti-infective active agent can be at least one selected from the group consisting of amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives.

A cardiovascular active agent can be at least one selected from the group consisting of inotropics, antiarrhythmics, antianginals, antihypertensives, antilipemics, and miscellaneous cardiovascular drugs.

A central nervous system active agent can be at least one selected from the group consisting of nonnarcotic analgesics or at least one selected from antipyretics, nonsteroidal anti-inflammatory drugs, narcotic or at least one opioid analgesics, sedative hypnotics, anticonvulsants, antidepressants, antianxiety drugs, antipsychotics, central nervous system stimulants, antiparkinsonians, and miscellaneous central nervous system drugs.

An autonomic nervous system agent can be at least one selected from the group consisting of cholinergics (parasympathomimetics), anticholinergics, adrenergics (sympathomimetics), adrenergic blockers (sympatholytics), skeletal muscle relaxants, neuromuscular blockers.

A respiratory tract active agent can be at least one selected from the group consisting of antihistamines, bronchodilators, expectorants or at least one antitussive, and miscellaneous respiratory drugs.

A GI tract active agent can be at least one selected from the group consisting of antacids or at least one adsorbents or at least one antiflatulents, digestive enzymes or at least one gallstone solubilizers, antidiarrheals, laxatives, antiemetics, and antiulcer drugs as well as the new irritable bowel molecules which operate as guanylate cyclase inhibitors (linaclotide).

A hormonal active agent can be at least one selected from the group consisting of corticosteroids, androgens or at least one anabolic steroids, estrogens or at least one progestins, gonadotropins, antidiabetic drugs or at least one glucagon, thyroid hormones, thyroid hormone antagonists, pituitary hormones, and parathyroid-like drugs.

An active agent for fluid and electrolyte balance can be at least one selected from the group consisting of diuretics, electrolytes or at least one replacement solution, acidifiers or at least one alkalinizer.

A hematologic active agent can be at least one selected from the group consisting of hematinics, anticoagulants, blood derivatives, thrombolytic enzymes.

An antineoplastic agent can be at least one selected from the group consisting of alkylating drugs, antimetabolites, antibiotic antineoplastics, antineoplastics that alter hormone balance, and miscellaneous antineoplastics.

An immunomodulation active agent can be at least one selected from the group consisting of immunosuppressants, vaccines or at least one toxoid, antitoxins or at least one antivenins, immune serums, biological response modifiers.

An ophthalmic, otic, and nasal active agent can be at least one selected from the group consisting of ophthalmic anti-infectives, ophthalmic anti-inflammatories, miotics, mydriatics, ophthalmic vasoconstrictors, miscellaneous ophthalmics, otics, and nasal active agents.

A topical active agent can be at least one selected from the group consisting of local anti-infectives, scabicides or at least one pediculicides, and topical corticosteroids. Amebicide or antiprotozoal can be at least one selected from atovaquone, chloroquine hydrochloride, chloroquine phosphate, metronidazole, metronidazole hydrochloride, and pentamidine isethionate.

An anthelmintic active agent can be at least one selected from the group consisting of mebendazole, pyrantel pamoate, and thiabendazole.

An antifungal agent can be at least one selected from the group consisting of amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin, and terbinafine hydrochloride.

An antimalarial agent can be at least one selected from the group consisting of chloroquine hydrochloride, chloroquine phosphate, doxycycline, hydroxychloroquine sulfate, mefloquine hydrochloride, primaquine phosphate, pyrimethamine, and pyrimethamine with sulfadoxine.

An antituberculotic or antileprotic agent can be at least one selected from the group consisting of clofazimine, cycloserine, dapsone, ethambutol hydrochloride, isoniazid, pyrazinamide, rifabutin, rifampin, rifapentine, and streptomycin sulfate.

An aminoglycoside agent can be at least one selected from the group consisting of amikacin sulfate, gentanicin sulfate, neomycin sulfate, streptomycin sulfate, and tobramycin sulfate.

The penicillin can be at least one selected from the group consisting of amoxcillin/clavulanate potassium, amoxicillin trihydrate, ampicillin, ampicillin sodium, ampicillin tribydrate, ampicillin sodium/sulbactam sodium, cloxacillin sodium, dicloxacillin sodium, mezlocillin sodium, nafcillin sodium, oxacillin sodium, penicillin G benzathine, penicillin G potassium, penicillin G procaine, penicillin G sodium, and penicillin V potassium.

The cephalosporin can be at least one selected from the group consisting of at least one of cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, ceffiroxime axetil, cefuroxime sodium, cephalexin hydrocllloride, cephalexin monohydrate, cephradine, and loracarbef.

The tetracycline can be at least one selected from the group consisting of demeclocycline hydrochloride, doxycycline calcium, doxycycline hyclate, doxycycline hydrochloride, doxycycline monobydrate, minocycline hydrochloride, and tetracycline hydrochloride. Sulfonamide can be at least one selected from co-trimoxazole, sulfadiazine, sulfamethoxazole, sulfisoxazole, and sulosoxazole acetyl. Fluoroquinolone can be at least one selected from alatrofloxacin mesylate, ciprofloxacin, enoxacin, levofloxacin, lomefloxacin hydrochloride, nalidixic acid, norfloxacin, ofloxacin, sparfloxacin, and trovafloxacin mesylate.

The fluoroquinolone can be at least one selected from the group consisting of alatrofloxacin mesylate, ciprofloxacin, enoxacin, levofloxacin, lomefloxacin hydrochloride, nalidixic acid, norfloxacin, ofloxacin, sparfloxacin, and trovafloxacin mesylate.

The antiviral active agent can be at least one selected from the group consisting of abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine, lamivodine/zidovodine, nelfinavir mesylate, nevirapine, oseltamivir phosphate, ribavirin, rimantadine hydrochloride, ritonavir, saquinavir, saquinavir mesylate, stavodine, valacyclovir hydrochloride, zalcitabine, zanamivir, and zidovudine.

A macroline anti-infective active agent can be at least one selected from the group consisting of azithromycin, clarithromycin, dirithromycin, erythromycin base, erythromycin estolate, erythromycin ethylsuccinate, erythromycin lactobionate, and erythromycin stearate.

An antiinfective active agent can also be at least one selected from the group consisting of aztreonam, bacitracin, chloramphenicol sodium sucinate, clindamycin hydrochloride, clindamycin palmitate 30 hydrochloride, clindamycin phosphate, imipenem and cilastatin sodium, meropenem, nitrofurantoin macrocrystals, nitrofurantoin microcrystals, quinupristin/dalfopristin, spectinomycin hydrochloride, trimethoprim, and vancomycin hydrochloride.

An inotropic active agent can be at least one selected from the group consisting of amrinone lactate, digoxin, and milrinone lactate.

An antiarrhythmic active agent can be at least one selected from the group consisting of adenosine, amiodarone hydrochloride, atropine sulfate, bretylium tosylate, diltiazem hydrochloride, disopyramide, disopyramide phosphate, esmolol hydrochloride, flecainide acetate, ibutilide fumarate, lidocaine hydrochloride, mexiletine hydrochloride, moricizine hydrochloride, phenytoin, phenytoin sodium, procainamide hydrochloride, propafenone hydrochloride, propranolol hydrochloride, quinidine bisulfate, quinidine gluconate, quinidine polygalacturonate, quinidine sulfate, sotalol, tocainide hydrochloride, and verapamil hydrochloride.

An antianginal active agent can be at least one selected from the group consisting of amlodipidine besylate, amyl nitrite, bepridil hydrochloride, diltiazem hydrochloride, isosorbide dinitrate, isosorbide mononitrate, nadolol, nicardipine hydrochloride, nifedipine, nitroglycerin, propranolol hydrochloride, verapamil, and verapamil hydrochloride.

An antihypertensive active agent can be at least one selected from the group consisting of acebutolol hydrochloride, amlodipine besylate, atenolol, benazepril hydrochloride, betaxolol hydrochloride, bisoprolol fumarate, candesartan cilexetil, captopril, carteolol hydrochloride, carvedilol, clonidine, clonidine hydrochloride, diazoxide, diltiazem hydrochloride, doxazosin mesylate, enalaprilat, enalapril maleate, eprosartan mesylate, felodipine, fenoldopam mesylate, fosinopril sodium, guanabenz acetate, guanadrel sulfate, guanfacine hydrochloride, hydralazine hydrochloride, irbesartan, isradipine, labetalol hydrchloride, lisinopril, losartan potassium, methyldopa, methyldopate hydrochloride, metoprolol succinate, metoprolol tartrate, minoxidil, moexipril hydrochloride, nadolol, nicardipine hydrochloride, nifedipine, nisoldipine, nitroprusside sodium, penbutolol sulfate, perindopril erbumine, phentolamine mesylate, pindolol, prazosin hydrochloride, propranolol hydrochloride, quinapril hydrochloride, ramipril, telmisartan, terazosin hydrochloride, timolol maleate, trandolapril, valsartan, and verapamil hydrochloride.

An antilipemic active agent can be at least one selected from the group consisting of atorvastatin calcium, cerivastatin sodium, cholestyramine, colestipol hydrochloride, fenofibrate (micronized), fluvastatin sodium, gemfibrozil, lovastatin, niacin, pravastatin sodium, simvastatin.

A cardiovascular active agent can be at least one selected from the group consisting of abciximab, alprostadil, arbutamine hydrochloride, cilostazol, clopidogrel bisulfate, dipyridamole, eptifibatide, midodrine hydrochloride, pentoxifylline, ticlopidine hydrochloride, and tirofiban hydrochloride.

An antihistamine active agent can be at least one selected from the group consisting of brompheniramine maleate, cetirizine hydrochloride, chlorpheniramine maleate, clemastine fumarate, cyproheptadine hydrochloride, diphenLydramine hydrochloride, fexofenadine hydrochloride, loratadine, promethazine hydrochloride, promethazine theoclate, and triprolidine hydrochloride.

A bronchodilator agent can be at least one selected from the group consisting of albuterol, albuterol sulfate, aminophylline, atropine sulfate, ephedrine sulfate, epinephrine, epinephrine bitartrate, epinephrine hydrochloride, ipratropium bromide, isoproterenol, isoproterenol hydrochloride, isoproterenol sulfate, levalbuterol hydrochloride, metaproterenol sulfate, oxtriphylline, pirbuterol acetate, salmeterol xinafoate, terbutaline sulfate, and theophylline.

An expectorant or antitussive agent can be at least one selected from the group consisting of benzonatate, codeine phosphate, codeine sulfate, dextramethorphan hydrobromide, diphenhydramine hydrochloride, guaifenesin, and hydromorphone hydrochloride. Respiratory active agent may be at least one selected from acetylcysteine, beclomethasone dipropionate, beractant, budesonide, calfactant, cromolyn sodium, dornase alfa, epoprostenol sodium, flunisolide, palivizumab, triamcinolone acetonide, zafirlukast, and zileuton.

An antacid, adsorbent, or antiflatulent agent can be at least one selected from the group consisting of aluminum carbonate, aluminum hydroxide, calcium carbonate, magaldrate, magnesium hydroxide, magnesium oxide, simethicone, and sodium bicarbonate.

A digestive enzyme or gallstone solubilizer active agent can be at least one selected from the group consisting of pancreatin, pancrelipase, and ursodiol.

An antidiarrheal active agent can be at least one selected from the group consisting of attapulgite, bismuth subsalicylate, calcium polycarbophil, diphenoxylate hydrochloride or atropine sulfate, loperamide, octreotide acetate, opium tincture, and opium tincure (camphorated). Laxative active agents may be at least one selected from bisocodyl, calcium polycarbophil, cascara sagrada, cascara sagrada aromatic fluidextract, cascara sagrada fluidextract, castor oil, docusate calcium, docusate sodium, glycerin, lactulose, magnesium citrate, magnesium hydroxide, magnesium sulfate, methylcellulose, mineral oil, polyethylene glycol or electrolyte solution, psyllium, senna, and sodium phosphates.

An antiemetic active agent can be at least one selected from the group consisting of chlorpromazine hydrochloride, dimenhydrinate, dolasetron mesylate, dronabinol, granisetron hydrochloride, meclizine hydrochloride, metocloproamide hydrochloride, ondansetron hydrochloride, perphenazine, prochlorperazine, prochlorperazine edisylate, prochlorperazine maleate, promethazine hydrochloride, scopolamine, thiethylperazine maleate, and trimethobenzamide hydrochloride.

An antiulcer active agent can be at least one selected from the group consisting of cimetidine, cimetidine hydrochloride, famotidine, lansoprazole, misoprostol, nizatidine, omeprazole, esomeprazole, rabeprozole sodium, rantidine bismuth citrate, ranitidine hydrochloride, and sucralfate.

A coricosteroid active agent can be at least one selected from the group consisting of betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate.

An androgen or anabolic steroid can be at least one selected from the group consisting of danazol, fluoxymesterone, methyltestosterone, nandrolone decanoate, nandrolone phenpropionate, testosterone, testosterone cypionate, testosterone enanthate, testosterone propionate, and testosterone transdermal system.

An estrogen or progestin agent can be at least one selected from the group consisting of esterified estrogens, estradiol, estradiol cypionate, estradiol/norethindrone acetate transdermal system, estradiol valerate, estrogens (conjugated), estropipate, ethinyl estradiol, ethinyl estradiol and desogestrel, ethinyl estradiol and ethynodiol diacetate, ethinyl estradiol and desogestrel, ethinyl estradiol and ethynodiol diacetate, ethinyl estradiol and levonorgestrel, ethinyl estradiol and norethindrone, ethinyl estradiol and norethindrone acetate, ethinyl estradiol and norgestimate, ethinyl estradiol and norgestrel, ethinyl estradiol and norethindrone and acetate and ferrous fumarate, levonorgestrel, medroxyprogesterone acetate, mestranol and norethindron, norethindrone, norethindrone acetate, norgestrel, and progesterone.

A gonadroptropin agent can be at least one selected from the group consisting of ganirelix acetate, gonadoreline acetate, histrelin acetate, and menotropins.

An antidiabetic active agent can be at least one selected from the group consisting of acarbose, chlorpropamide, glimepiride, glipizide, glucagon, glyburide, insulins, metformin hydrochloride, miglitol, pioglitazone hydrochloride, repaglinide, rosiglitazone maleate, and troglitazone.

A thyroid hormone active agent can be at least one selected from the group consisting of levothyroxine sodium, liothyronine sodium, liotrix, and thyroid.

A thyroid hormone antagonist active agent can be at least one selected from the group consisting of methimazole, potassium iodide, potassium iodide (saturated solution), propylthiouracil, radioactive iodine (sodium iodide), and strong iodine solution.

A pituitary hormone active agent can be at least one selected from the group consisting of corticotropin, cosyntropin, desmophressin acetate, leuprolide acetate, repository corticotropin, somatrem, somatropin, and vasopressin.

A parathyroid-like active agent can be at least one selected from the group consisting of calcifediol, calcitonin (human), calcitonin (salmon), calcitriol, dihydrotachysterol, and etidronate disodium.

A diuretic agent can be at least one selected from the group consisting of acetazolamide, acetazolamide sodium, amiloride hydrochloride, bumetanide, chlorthalidone, ethacrynate sodium, ethacrynic acid, furosemide, hydrochlorothiazide, indapamide, mannitol, metolazone, spironolactone, torsemide, triamterene, and urea.

An electrolyte or replacement solution active agent can be at least one selected from the group consisting of calcium acetate, calcium carbonate, calcium chloride, calcium citrate, calcium glubionate, calcium gluceptate, calcium gluconate, calcium lactate, calcium phosphate (dibasic), calcium phosphate (tribasic), dextran (high-molecular-weight), dextran (lowmolecular-weight), hetastarch, magnesium chloride, magnesium sulfate, potassium acetate, potassium bicarbonate, potassium chloride, potassium gluconate, Ringer's injection, Ringer's injection (lactated), and sodium chloride.

A hematinic active agent can be at least one selected from the group consisting of ferrous fumarate, ferrous gluconate, ferrous sulfate, ferrous sulfate (dried), iron dextran, iron sorbitol, polysaccharide iron complex, sodium ferric gluconate complex.

An anticoagulant active agent can be at least one selected from the group consisting of ardeparin sodium, dalteparin sodium, danaparoid sodium, 15 enoxaparin sodium, heparin calcium, heparin sodium, and warfarin sodium.

A blood derivative agent can be at least one selected from the group consisting of albumin 5%, albumin 25%, antihemophilic factor, anti inhibitor coagulant complex, antithrombin m (human), factor IX (human), factor IX complex, and plasma protein fractions.

A thrombolytic enzyme active agent can be selected from the group consisting of alteplase, anistreplase, reteplase (recombinant), streptokinase, urokinase.

An alkylating active agent can be at least one selected from the group consisting of busulfan, carboplatin, carmustine, chlorambucil, cisplatin, cyclophosphamide, ifosfamide, lomustine, mechl oreth amine hydrochloride, melphalan, melphal an hydrochloride, streptozocin, temozolomide, thiotepa.

An antimetabolite agent can be selected from the group consisting of capecitabine, cladribine, cytarabine, floxuridine, fludarabine phosphate, fluorouracil, hydroxyurea, mercaptopurine, methotrexate, methotrexate sodium, thioguanine.

An antibiotic antineoplastic agent can be selected from the group consisting of bleomycin sulfate, dactinomycin, daunorubicin citrate liposomal, daunorubicin hydrochloride, doxorubicin hydrochloride, doxorubicin hydrochloride liposomal, epirubicin hydrochloride, idaubicin hydrochloride, mitomycin, pentostatin, plicamycin, and valrubicin.

An antineoplastic agent can be selected from the group consisting of anastrozole, bicalutamide, estramustine phosphate sodium, exemestane, flutamide, goserelin acetate, letrozole, leuprolide acetate, megestrol acetate, nilutamide, tamoxifen citrate, testolactone, toremifene citrate, asparaginase, bacillus Calmette-Guerin (BCG), dacarbazine, docetaxel, etoposide, etoposide phosphate, gemcitabine hydrochloride, irinotecan hydrochloride, mitotane, mitoxantrone hydrochloride, paclitaxel, pegaspargase, porfimer sodium, procarbazine hydrochloride, rituximab, teniposide, topotecan hydrochloride, trastuzumab, tretinoin, vinblastine sulfate, vincristine sulfate, and vinorelbine tartrate.

An immunosuppressant active agent can be at least one selected from the group consisting of azathioprine, basiliximab, cyclosporine, daclizumab, lymphocyte immune globulin, muromonab-CD3, mycophenolate mofetil, mycophenolate mofetil hydrochloride, sirolimus, inflixamab, rituximab, entanerecept, certalizumab, adalimumab, tocilizumab, golimumab and tacrolimus.

A vaccine or toxoid active agent can be at least one selected from the group consisting of BCG vaccine, cholera vaccine, diphtheria and tetanus toxoids (adsorbed), diphtheria and tetanus toxoids and acellular pertussis vaccine adsorbed, diphtheria and tetanus toxoids and whole-cell pertussis vaccine, Haemophilius b conjugate vaccines, hepatitis A vaccine (inactivated), hepatisis B vaccine (recombinant), influenza virus vaccine 1999-2000 trivalent types A & B (purified surface antigen), influenza virus vaccine 1999-2000 trivalent types A & B (subvirion or purified subvirion), influenza virus vaccine 1999-2000 trivalent types A & B (whole virion), Japanese encephalitis virus vaccine (inactivated), influenza H1N1 vaccine, Lyme disease vaccine (recombinant OspA), measles and mumps and rubella virus vaccine (live), measles and mumps and rubella virus vaccine (live attenuated), measles virus vaccine (live attenuated), meningococcal polysaccharide vaccine, mumps virus vaccine (live), plague vaccine, pneumococcal vaccine (polyvalent), poliovirus vaccine (inactivated), poliovirus vaccine (live, oral, trivalent), rabies vaccine (adsorbed), rabies vaccine (human diploid cell), rubella and mumps virus vaccine (live), rubella virus vaccine (live, attenuated), tetanus toxoid (adsorbed), tetanus toxoid (fluid), typhoid vaccine (oral), typhoid vaccine (parenteral), typhoid Vi polysaccharide vaccine, varicella virus vaccine, and yellow fever vaccine.

An antitoxin or antivenin (or antivenom) active agent can be at least one selected from the group consisting of black widow spider antivenin, Crotalidae antivenom (polyvalent), diphtheria antitoxin (equine), and Micrurus fulvius antivenin).

An immune serum active agent can be at least one selected from the group consisting of cytomegalovirus immune globulin, hepatitis B immune globulin (human), immune globulin intramuscular, immune globulin intravenous, rabies immune globulin (human), respiratory syncytial virus immune globulin intravenous (human), Rho(D) immune globulin (human), Rho(D) immune globulin intravenous (human), tetanus immune globulin (human), and varicella-zoster immune globulin. Biological response modifiers can be at least one selected from aldesleukin, erythropoetin alfa, filgrastim, glatiramer acetate for injection, interferon alfacon-1, interferon alfa-2a (recombinant), interferon alfa-2b (recombinant), interferon beta-1a, interferon beta-1b (recombinant), interferon gamma-1b, levamisole hydrochloride, oprelvekin, and sargramostim.

An ophthalmic anti-infective agent can be selected form the group consisting of bacitracin, chloramphenicol, ciprofloxacin hydrochloride, erythromycin, gentamicin sulfate, ofloxacin 0.3%, polymyxin B sulfate, sulfacetamide sodium 10%, sulfacetamide sodium 15%, sulfacetamide sodium 30%, tobramycin, vidarabine. Ophthalmic anti-inflammatory active agents may be at least one selected from dexamethasone, 5 dexamethasone sodium phosphate, diclofenac sodium 0.1%, fluorometholone, flurbiprofen sodium, ketorolac tromethamine, prednisolone acetate, and prednisolone sodium phosphate.

A Miotic agent can be at least one selected from the group consisting of acetylocholine chloride, carbachol (intraocular), carbachol (topical), echothiophate iodide, pilocarpine, pilocarpine hydrochloride, and pilocarpine nitrate. Mydriatic active agents may be at least one selected from atropine sulfate, cyclopentolate hydrochloride, epinephrine hydrochloride, epinephryl borate, homatropine hydrobromide, phenylephrine hydrochloride, scopolamine hydrobromide, and tropicamide. Ophthalmic vasoconstrictors may be at least one selected from naphazoline hydrochloride, oxymetazoline hydrochloride, and tetrahydrozoline hydrochloride.

An ophthalmic agent can be at least one selected from the group consisting of apraclonidine hydrochloride, betaxolol hydrochloride, brimonidine tartrate, carteolol hydrochloride, dipivefrin hydrochloride, dorzolamide hydrochloride, emedastine difumarate, fluorescein sodium, ketotifen fumarate, latanoprost, levobunolol hydrochloride, metipranolol hydrochloride, sodium chloride (hypertonic), and timolol maleate.

An otic (ear) active agent can be at least one selected from the group consisting of boric acid, carbamide peroxide, chloramphenicol, and triethanolamine polypeptide oleate-condensate.

A nasal active agent can be at least one selected from the group consisting of beclomethasone dipropionate, budesonide, ephedrine sulfate, epinephrine hydrochloride, flunisolide, fluticasone propionate, naphazoline hydrochloride, oxymetazoline hydrochloride, phenylephrine hydrochloride, tetrabydrozoline hydrochloride, triamcinolone acetonide, and xylometazoline hydrochloride.

An anti-infective agent can also be at least one selected from the group consisting of acyclovir, amphotericin B., azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. Scabicide or pediculicide active agents may be at least one selected from crotamiton, lindane, permethrin, and pyrethrins.

A corticosteroid in all forms (IV, oral, SQ, IM, topical, suppository, intranasal, intraocular, tympanic, transdermal) may be at least one selected from the group consisting of betamethasone dipropionate, betamethasone valerate, clobetasol propionate, desonide, desoximetasone, diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide.

Additional active agents, or classes of active agents, include Tumor necrosis factor (TNF) antagonists (e.g., a TNF chemical or protein antagonist), TNF monoclonal or polyclonal antibody or fragment, a soluble TNF receptor (e.g., p55, p70 or p85) or fragment, fusion polypeptides thereof, or a small molecule TNF antagonist (e.g., TNF binding protein I or II (TBP-1 or TBP-II)), nerelimonmab, infliximab, enteracept, CDP-571, 2 5 CDP-870, afelimomab, lenercept, and the like), bremelanotide, banana spider venom (peptide K), various vitamins and minerals, etc.

Active agents can further include an antirheumatic (e.g., methotrexate, auranofin, aurothioglucose, azathioprine, etanercept, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM 3 5 CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrrne, an asthma medication, a beta agonist, an inhaled steroid, a leukotriene inhibitor, a methylxanthine, a cromolyn, an epinephrine or analog, dornase alpha, a cytokine or a cytokine antagonist. Non-limiting examples of such cytokines include, but are not limited to, any of the interleukins, including IL-1 to IL-23.

Active ingredients for over-the-counter cosmetic purposes can include GRAS compounds including, but not limited to, caffeine, Deionized Water, Simmondsia, Chinensis Seed Oil, Acetyl Hexpeptide-8, Hydroxyethyl Acrylate, Sodium Acryloyldimethyltaurate Copolymer, Squalane, Polysorbate 60, Ubiquinone, Cyclomethicone, Dimethiconol, Ethylhexyl Cocoate, Glycine Soja Oil, Retinol, Lecithin, Glycolipids, Camellia Sinensis Leaf Extract, glycerin, Dipeptide Diaminobutyroyl Benzylamide Diacetate, Sodium Hyaluronate, Phenoxyethanol, Caprylyl Glycol, Potassium Sorbate, aqua, Hexylene glycol, Caprylic/Capric triglyceride, Triethanolamine, Rosa Canina Extract, Malus Domestica Fruit Cell culture, Xanthan gum, Panax ginseng Extract, Scorbic acid, Punica Granatum Extract, Melaleuca Alternifolia Leaf Oil, Frangrance, Polymethylsilsesquinoxane, Cyclopentasiloxane, Dimethicone, Polysilicone-11, Butylene glycol, Decyl glucoside, Macadamia ternifolia Seed Oil, Macelignan, Tocopherol, Acetyl hexapeptide-8, Hydroxyethyl acrylate, Sodium Acryloyldimethyltaurate Copolymer, Squalane, Polysorbate 60, Sodium Hyaluronate, silica, Phenoxyethanol, Caprylyl Glycol, Potassium Sorbate, Hexylene glycol, seaweed extract, Plankton Extract, Sea Buckhorn Extract, Watercress Extract, Marine Algae, Hyaluronic Acid, Phenoxyethanol, Caprylyl glycol, Potassium sorbate, Hexylene Glycol, Hyaluronic Acid, Algae Extract, Pseudoalteromonas Ferment Extract, Hydrolyzed Wheat Protein, Hydrolyzed Soy Protein, Tripeptide-10 Cirulline, Tripeptide-1, Lecihin, Xanthan Gum, Carbomer, Triehanolamine, Malus Domestica Acetyl Hexapeptide-8, Pullulan, Henoxyethanol, Caprylyl Glycol, Potassium Sorbate, Hexylene Glycol, tranilast, bromelain, rhamnose, cats claw, eucommia boswellic acid, resveratrol, lipoic acid, and Butchers broom rhizome.

Active ingredients for medical cosmetic purposes, including inert fillers, can include hyaluronic acid, silicone and collagen, lidocaine, saline, botulism toxin, calcium hydroxylapatite, poly-L-lactic acid (“PPLLA”), polymethylmethacrylate beads (“PMMA microspheres), calcium sulfate and caffeine.

Active ingredients can also include sterile fluid.

The amount of active agent can range from about 0.01 mg to about 1000 mg. Specific active agent dosages within a single formulation can include, but are not limited to about 0.01, 0.02, 0.05, 0.1, 0.5, 1, 2, 5, 10, 25, 30, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, and about 1000 mg. The active agent may comprise, but is not limited to, about 0.01 to 95% of the total composition weight of the delivery fluid volume, such as about 0.01, 0.02, 0.05, 0.1, 0.5, 1, 2, 5, 10, 20, 30, 40, 50, 60, 70, 80, 90 or about 95% of the total composition by weight.

The formulations of active ingredients delivered by the delivery system described herein may be in any suitable shape, size or form. Advantageously, the formulations described herein can be designed such that they provide immediate release of the treating agent. “Immediate release” refers to the release of the active agent at the time of administration. Thus, the release of the active agent may occur at the moment of contact with the patient. Alternatively, the formulations of the active ingredients described herein can be designed to delay the release of the active agent as would be understood by one skilled in the art. Such sustained and/or timed release formulations may be made by sustained release means of delivery devices that are well known to those of ordinary skill in the art. These active ingredient compositions can be used to provide slow or sustained release of one or more of the active compounds using, for example, hydroxypropylmethyl cellulose, other polymer matrices, gels, permeable membranes, osmotic systems, multilayer coatings, microparticles, liposomes, microspheres, or the like.

Suitable sustained release formulations known to those skilled in the art, including those described herein, may be readily selected for use with the active ingredient compositions delivered by the invention. Thus, the release of the active agent may occur after a period of time from the moment of contact with the patient. Additionally formulations may include a combination of delayed release and immediate release.

### Some Aspects of the Present Invention

As used herein, the term “non-invasive” is intended to describe a proprietary vehicle as delivering an active agent from an external site (i.e., mucocutaneous) to an internal site without disruption, or at only a nanoscale of disruption of the membrane interface.

In one aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system includes at least one composition that includes at least one of the following: a sterile fluid with or without a therapeutic agent.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system includes at least a salty fluid that is ionizable or ionized.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system includes at least a amino acid, protein, sugar, a detergent, or a combination thereof.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system includes at least a pH buffered fluid.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein at least one component is a micro-syringe assembly and includes a reservoir and/or a continuous fluid input system.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system is in the form of a tablet, a reusable device, a patch, gel, cream or lotion.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system can be administered, topically to the skin or through a mucosal membrane.

In another aspect of the present invention, there is provided a painless, non-invasive delivery system for delivering a therapeutic agent across an epithelial membrane, the system comprising: (a) an effective amount of a therapeutic agent; and (b) at least one vehicle for facilitating delivery of the agent across the epithelial membrane, the vehicle being selected from the following: (i) a sterile fluid, such as normal saline; and (ii) the use of carbon nanotube ring structures in a delivery system, either free-standing or in an array formation affixed to or embedded in a substrate assembly including scaffolding, and possibly hollow; and (iii) nanosyringes (microsyringes) of appropriate lengths arranged in either an array formation or free-standing, affixed to or embedded in a substrate assembly, wherein the system further comprises a patch-like design with an adhesive component to stabilize the micro-syringes during penetration of the skin or mucosal membrane.

In another aspect of the present invention, there is provided a method of treating a disease or condition in a mammal, the method comprising: administering a non-invasive delivery system that comprises an effective amount of an agent being delivered across an epithelial membrane via a micro-syringe-based assembly.

In another aspect of the present invention, there are provided novel methods and apparatus for delivering an active agent to a patient, the methods and apparatus comprising the use of: (a) an effective amount of an agent; and (b) at least one vehicle for facilitating delivery of the active ingredient across the epithelial membrane, the vehicle being selected from the following: (i) a carbon nanotube-based micro-syringe assembly; and (ii) a sterile fluid incorporated during or after the manufacture of the painless, non-invasive delivery system for delivering an agent into a patient by penetration through an epithelial membrane.

In another aspect of the present invention, there are provided novel methods and apparatus for delivering an active agent to a patient, the methods and apparatus comprising the use of: (a) an effective amount of an agent; and (b) at least one vehicle for facilitating delivery of the active ingredient across the epithelial membrane, the vehicle being selected from the following: (i) a carbon nanotube-based micro-syringe assembly; and (ii) a sterile fluid incorporated during or after the manufacture of the painless, non-invasive delivery system for delivering an agent into a patient by penetration through an epithelial membrane, wherein the delivery system is for use in treating a condition that requires immediate, sustained or delayed release of the active agent.

In another aspect of the present invention, there are provided novel methods and apparatus for delivering an active agent to a patient, the methods and apparatus comprising the use of: (a) an effective amount of an agent; and (b) at least one vehicle for facilitating delivery of the active ingredient across the epithelial membrane, the vehicle being selected from the following: (i) a carbon nanotube-based micro-syringe assembly; and (ii) a sterile fluid incorporated during or after the manufacture of the painless, non-invasive delivery system for delivering an agent into a patient by penetration through an epithelial membrane, wherein the delivery system is for use in treating a condition that requires immediate, sustained or delayed release of the active agent, and further wherein the delivery system is for use in treating anaphylactic shock, diabetes (both high and low blood sugars), ischemic heart disease or trauma.

In another aspect of the present invention, there is provided a painless, non-invasive delivery system that comprises an effective amount of an agent and at least one enabler so as to facilitate absorption of the agent into the epithelial membrane, the delivery system being for use in treating a condition that requires immediate, sustained or delayed release of said active agent.

In another aspect of the present invention, there is provided a painless, non-invasive delivery system that comprises an effective amount of an agent and at least one enabler so as to facilitate absorption of the agent into the epithelial membrane, the delivery system being for use in treating a condition that requires immediate, sustained or delayed release of said active agent, wherein the condition to be treated is selected from anaphylactic shock and diabetes.

In another aspect of the present invention, there is provided a non-invasive delivery vehicle for use in filtration through the gradation of CNT lumen diameter either in an increasing, decreasing or variable pattern.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. Apparatus for delivering an active agent to a patient, said apparatus comprising:
a carrier comprising a flexible concave member; and
a syringe mounted to said flexible concave member, said syringe comprising:
a chamber having a proximal end and a distal end and containing an active agent to be delivered to a patient, wherein said distal end of said chamber comprises a wafer substrate having a plurality of openings formed therein and having plurality of hollow fibers extending distally therefrom;
a locking mechanism disposed in telescoping relation with said chamber;
a support band disposed in telescoping relation with said locking mechanism;
one or more retention mechanisms that releasably secure said locking mechanism to said support band; and
a plunger having a proximal end and a distal end and a telescoping rod disposed therebetween, wherein said distal end comprises a plunger disc disposed in said chamber and wherein said proximal end of said plunger comprises a comfort top that communicates with said flexible concave member of said carrier; and
a spring disposed between said comfort top and said proximal end of said chamber so as to bias said comfort top proximally away from said proximal end of said chamber;
wherein when a distally-directed force is applied to said comfort top, said force overcomes said one or more retention mechanisms, moves said chamber and said locking mechanism as a unit distally and telescopically relative to said support band thereby deploying said hollow fibers distally beyond said support band and into the patient, and further wherein continued application of the distally-directed force to said plunger top overcomes the proximal bias of said spring and causes said plunger disc to move distally and push the active agent out of said chamber through said openings in said wafer substrate, through said hollow fibers and into the patient.

2. Apparatus according to claim 1 wherein a peel-away strip extends across the bottom of flexible concave member thereby enclosing said syringe inside said concave member.

3. Apparatus according to claim 2 wherein the volume of the concavity not occupied by said syringe is filled with a gel.

4. Apparatus according to claim 1 wherein said hollow fibers are provided in such numbers and in closely-spaced relation to one another so as to create a self-supporting meta-structure.

5. Apparatus according to claim 1 wherein said hollow fibers comprise carbon nanotubes.

6. Apparatus according to claim 1 wherein said hollow fibers comprise tubular structures.

7. Apparatus according to claim 1 wherein said one or more retention mechanisms comprise shear tabs.

8. Apparatus according to claim 1 wherein the plunger disc is formed of silicone.

9. Apparatus according to claim 1 wherein each of said plurality of hollow fibers is long enough to penetrate the skin of the patient and narrow enough to avoid causing pain to the patient.

10. Apparatus according to claim 1 wherein a sufficient number of hollow fibers are provided so as to deliver the desired quantity of the active agent from said chamber to the patient within a desired time.

11. A method for delivering an active agent to a patient, said method comprising:
providing apparatus for delivering an active agent to a patient, said apparatus comprising:
a carrier comprising a flexible concave member; and
a syringe mounted to said flexible concave member, said syringe comprising:
a chamber having a proximal end and a distal end and containing an active agent to be delivered to a patient, wherein said distal end of said chamber comprises a wafer substrate having a plurality of openings formed therein and having plurality of hollow fibers extending distally therefrom;
a locking mechanism disposed in telescoping relation with said chamber;
a support band disposed in telescoping relation with said locking mechanism;
one or more retention mechanisms that releasably secure said locking mechanism to said support band; and
a plunger having a proximal end and a distal end and a telescoping rod disposed therebetween, wherein said distal end comprises a plunger disc disposed in said chamber and wherein said proximal end of said plunger comprises a comfort top that communicates with said flexible concave member of said carrier; and
a spring disposed between said comfort top and said proximal end of said chamber so as to bias said comfort top proximally away from said proximal end of said chamber;
wherein when a distally-directed force is applied to said comfort top, said force overcomes said one or more retention mechanisms, moves said chamber and said locking mechanism as a unit distally and telescopically relative to said support band thereby deploying said hollow fibers distally beyond said support band and into the patient, and further wherein continued application of the distally-directed force to said plunger top overcomes the proximal bias of said spring and causes said plunger disc to move distally and push the active agent out of said chamber through said openings in said wafer substrate, through said hollow fibers and into the patient;
disposing said carrier against the skin of the patient at a desired location;
applying a distally-directed force to said comfort top to overcome said one or more retention mechanisms, whereby to move said chamber and said locking mechanism as a unit distally and telescopically relative to said support band thereby deploying said hollow fibers into the skin of the patient;
continuing the application of said distally-directed force to move said plunger disc distally so as to push the active agent out of said chamber through said openings in said wafer substrate, through said hollow fibers and into the patient.

12. Apparatus for delivering an active agent to a patient, said apparatus comprising:
a flexible carrier comprising a concavity; and
a syringe mounted within said concavity of said flexible carrier, said syringe comprising:
a hollow base comprising a distal end and a proximal end;
a cap movably mounted to said proximal end of said hollow base;
a spring disposed between said distal end of said hollow base and said cap, said spring being configured to proximally bias said cap;
a wafer substrate comprising a distal surface and a proximal surface, and a plurality of openings extending between said distal surface and said proximal surface, said wafer substrate being movably disposed within said hollow base;
a plurality of nano-needles extending distally from said distal surface of said wafer substrate, wherein each of said nano-needles comprises a distal end and a proximal end, and a lumen extending therebetween, and further wherein said each lumen of each of said plurality of nano-needles is aligned with said openings formed in said wafer substrate;
a rod having a distal end and a proximal end, wherein said distal end of said rod is mounted to, and extends between, said wafer substrate and said cap;
a needle support plate mounted to the distal end of said hollow base, said needle support plate comprising a plurality of openings sized to receive a plurality of nano-needles therein;
a timing ring having a distal end and a proximal end, and a plunger disc mounted to said distal end of said timing ring, wherein said timing ring and said plunger disc are disposed coaxially about said rod intermediate said cap and said wafer substrate, whereby to define a chamber between said plunger disc and said wafer substrate for containing the active agent which is to be delivered to a patient, and further wherein said timing ring is configured to selectively move longitudinally relative to said rod and is configured to selectively rotate relative to said rod;
a torsional spring disposed between said cap and said distal end of said timing ring;
a locking ring mounted to said timing ring and to said hollow base, said locking ring being configured to selectively permit said timing ring to rotate relative to said rod;
wherein when said cap is moved distally, (i) said wafer substrate moves distally so as to project said plurality of nano-needles through said openings formed in said needle support plate and into the patient, and (ii) said locking ring releases said timing ring, whereby to allow said torsional spring to bias said timing ring and said plunger disc distally and thereby force the active agent contained in said chamber into said openings formed in said substrate, through said lumens of said plurality of nano-needles, and into the patient.

13. Apparatus according to claim 12 wherein a peel-away strip extends across the bottom of flexible carrier sealing said syringe within said concavity.

14. Apparatus according to claim 13 wherein the volume of the concavity not occupied by said syringe is filled with a gel.

15. Apparatus according to claim 12 further comprising a spring-biased needle guide plate disposed between said distal surface of said wafer substrate and said needle support plate, wherein said spring-biased needle guide plate comprises a plurality of openings sized to receive said plurality of nano-needles so as provide lateral support thereto.

16. Apparatus according to claim 12 wherein said nano-needles comprise carbon nanotubes.

17. Apparatus according to claim 12 wherein said nano-needles comprise tubular structures.

18. Apparatus according to claim 12 further comprising a cycle indicator mounted to said locking ring for indicating the cycle status of said syringe.

19. Apparatus according to claim 12 wherein each of said plurality of nano-needles is long enough to penetrate the skin of the patient and narrow enough to avoid causing pain to the patient.

20. Apparatus according to claim 12 wherein a sufficient number of nano-needles are provided so as to deliver the desired quantity of the active agent from said chamber to the patient within a desired time.

21. A method for delivering an active agent to a patient, said method comprising:
providing apparatus for delivering an active agent to a patient, said apparatus comprising:
a flexible carrier comprising a concavity; and
a syringe mounted within said concavity of said flexible carrier, said syringe comprising:
a hollow base comprising a distal end and a proximal end;
a cap movably mounted to said proximal end of said hollow base;
a spring disposed between said distal end of said hollow base and said cap, said spring being configured to proximally bias said cap;
a wafer substrate comprising a distal surface and a proximal surface, and a plurality of openings extending between said distal surface and said proximal surface, said wafer substrate being movably disposed within said hollow base;
a plurality of nano-needles extending distally from said distal surface of said wafer substrate, wherein each of said nano-needles comprises a distal end and a proximal end, and a lumen extending therebetween, and further wherein said each lumen of each of said plurality of nano-needles is aligned with said openings formed in said wafer substrate;
a rod having a distal end and a proximal end, wherein said distal end of said rod is mounted to, and extends between, said wafer substrate and said cap;
a needle support plate mounted to the distal end of said hollow base, said needle support plate comprising a plurality of openings sized to receive a plurality of nano-needles therein;
a timing ring having a distal end and a proximal end, and a plunger disc mounted to said distal end of said timing ring, wherein said timing ring and said plunger disc are disposed coaxially about said rod intermediate said cap and said wafer substrate, whereby to define a chamber between said plunger disc and said wafer substrate for containing the active agent which is to be delivered to a patient, and further wherein said timing ring is configured to selectively move longitudinally relative to said rod and is configured to selectively rotate relative to said rod;
a torsional spring disposed between said cap and said distal end of said timing ring;
a locking ring mounted to said timing ring and to said hollow base, said locking ring being configured to selectively permit said timing ring to rotate relative to said rod;
wherein when said cap is moved distally, (i) said wafer substrate moves distally so as to project said plurality of nano-needles through said openings formed in said needle support plate and into the patient, and (ii) said locking ring releases said timing ring, whereby to allow said torsional spring to bias said timing ring and said plunger disc distally and thereby force the active agent contained in said chamber into said openings formed in said substrate, through said lumens of said plurality of nano-needles, and into the patient;
disposing said flexible carrier against the skin of the patient at a desired location;
applying a distal force so as to move said cap distally, whereby to (i) advance said plurality of nano-needles into the skin of the patient, and (ii) force the active agent contained in said chamber into said openings formed in said substrate, through said lumens of said plurality of nano-needles, and into the patient; and
allowing said cap to move proximally under the power of said spring disposed between said distal end of said hollow base and said cap, whereby to move said wafer substrate and said plurality of nano-needles proximally, whereby to withdraw said nano-needles from the skin of the patient.

22. A nano-needle comprising a plurality of carbon nanotubes having a matrix material filling the interstitial spaces between said carbon nanotubes.

23. A method for making a nano-needle comprising a plurality of carbon nanotubes, said method comprising:
providing a wafer substrate having one or more openings extending therethrough;
depositing a catalyst around the periphery of said one or more openings extending through said wafer substrate;
activating said catalyst so that said catalyst forms islands around the periphery of said one or more openings;
growing a plurality of carbon nanotubes from said islands;
applying a matrix material to the interstitial spaces between said carbon nanotubes so as to form a hollow nano-needle having a diameter that is roughly defined by the periphery of said one or more openings.
